(19) United States
(12) Patent Application Publication
(10) Pub. No.: US 2003/0119775 A1
(43) Pub. Date: Jun. 26, 2003

(54) Respiration uncoupling protein

(75) Inventors: Richard S. Surwit, Chapel Hill, NC (US); Sheila A. Collins, Durham, NC (US); Craig H. Warden, Davis, CA (US); Michael F. Seldin, Davis, CA (US); Daniel Ricquier, Carrieres-sous-Poissy (FR); Frederic Bouillaud, Meudon la Foret (FR)

Correspondence Address: NIXON & VANDERHYE, PC, 1100 N GLEBE ROAD, 8TH FLOOR, ARLINGTON, VA, 22201-4714, US

(73) Assignees: DUKE UNIVERSITY; REGENTS OF THE UNIVERSITY OF CALIFORNIA; CENTRE NATIONAL DE LA RECHERCHE SCIENTIFIQUE

(21) Appl. No.: 10/265,689

(22) Filed: Oct. 8, 2002

Related U.S. Application Data
(63) Continuation of application No. 09/353,645, filed on Jul. 15, 1999, now abandoned.
Application No. 09/353,645 is a continuation of application No. PCT/US97/06864, filed on Apr. 22, 1997.
(60) Provisional application No. 60/034,960, filed on Jan. 15, 1997.

(51) Int. Cl. A61K 48/00; C12N 9/64

(52) U.S. Cl. 514/44; 435/226

(57) ABSTRACT

The present invention relates, in general, to a protein linked to cell respiration, thermogenesis, obesity and hyperinsulinemia and, in particular, to a protein designated uncoupling protein-2 (UCP2) and to nucleic acid sequences encoding same. The invention also relates to diagnostic methodologies based, for example, on a determination of levels of UCP2 expression. Further, the invention relates to therapies involving modulating UCP2 expression and/or activity. In addition, the present invention relates to methods of screening compounds for their suitability for use in such therapies.

The entire content of Provisional Application No. 60/034,960, filed Jan. 15, 1997, is incorporated herein by reference.

### TECHNICAL FIELD

The present invention relates, in general, to a protein linked to cell respiration, metabolic rate, thermogenesis, obesity and hyperinsulinemia and, in particular, to a protein designated uncoupling protein-2 (UCP2) and to nucleic acid sequences encoding same. The invention also relates to diagnostic methodologies based, for example, on a determination of levels of UCP2 expression. Further, the invention relates to therapies involving modulating UCP2 expression and/or activity. In addition, the present invention relates to methods of screening compounds for their suitability for use in such therapies.

### BACKGROUND

Obesity is a major health problem in most industrialized countries, including the United States. The seriousness of the pathologies associated with obesity, including type II diabetes and hypertension, has prompted numerous studies directed at determining the molecular basis for obesity. One result of such studies is the demonstration that genetically inherited characteristics may play a significant role in the development of the disorder.

Recent reports indicate that obesity may be associated with an energy imbalance. The energy balance of an individual is dependent upon both the energy intake (food) and the energy output (corresponding to resting metabolic rate metabolism, physical exercise and heat production). One hypothesis is that decreased resting metabolic rate and/or thermogenesis contributes to the development of obesity. Results of studies relating to uncoupling protein-1 (UCP1) demonstrate that such is the case in rodents (Ricquier et al, FASEB J. 5:2237 (1991); Muzin et al, Biochem. J. 261:721 (1989); Ricquier et al, J. Biol. Chem. 261:13905 (1986); Collins et al, Endocrinol. 138:405 (1997)).

UCP1 creates a pathway that allows dissipation of the electrochemical gradient of protons across the inner mitochondrial membrane in brown adipose tissue, without coupling to any other energy consuming process. This results in generation of heat and dissipation of calories. This process has been implicated in the regulation of body temperature, body composition and glucose metabolism in animals. However, UCP1-containing brown adipose tissue is unlikely to play a role in weight regulation in adult animals and humans living in a thermoneutral environment since there is little brown adipose tissue present.

The present invention relates to a novel uncoupling protein, designated UCP2. The chromosomal location of the UCP2 gene, and uncoupling activity and tissue distribution of UCP2 are consistent with a role for this protein in diabetes and obesity.

### BRIEF DESCRIPTION OF THE DRAWINGS

FIGS. 1A-G. A. Human UCP2 amino acid sequence. (Human UCP2 sequence is GenBank accession U76367.) The three mitochondrial carrier protein motifs present in UCP1 and conserved in UCP2 are underlined. B. Nucleotide sequence encoding human UCP2 amino acid sequence shown in A, including the stop codon. C. Map of the human UCP2 gene. D. Subcloned fragments of hUCP2-g1. pSU04, pSUB14sp6, pSUB 14t7, pSUB23 sp6, pSUB23t7 are non localized fragments of the human UCP2 gene. pSUB 12 SP6 corresponds to the 3′ end of the gene; pSUB5t7 corresponds to the promoter region; ex 12 identifies a genomic region containing exon 1 and 2 plus intronic regions; ex 34 identifies a genomic region containing exon 3 and 4 plus intronic regions; and ex 56 identifies a genomic region containing exon 5 and 6 plus intronic regions. Cloning strategy: A mouse UCP cDNA was first cloned using rat UCP1 cDNA to screen a mouse muscle cDNA library (lambda GT11, Clontech). Primers were derived from the mouse UCP2 cDNA to amplify a genomic human fragment corresponding to exons 1 and 2, plus introns 1 and 2 of the human UCP2 gene. This partial genomic fragment was then used to clone the entire human UCP2 gene, hUCP2-g1. The library screened was a human genomic library from Clontech (cat# HL 1067j; lot #45003) made in the EMBL3 SP6/T7 cloning vector with E. coli K802 as host strain. The DNA source was human placenta. E. Consensus sequence for the entire human expressed UCP2. The entire expressed sequence (mRNA) is 1612 bp long. The coding sequence extends from bp 345 to 1275 of this sequence. The clones hUCP2-5′ and hUCP2-3′, from which the consensus sequence was derived, were isolated from the human lung Marathon library from Clontech (Palo Alto, Calif.; catalog #7408-1). They were isolated by Random Amplification of cDNA Ends (RACE). 5′ and 3′ RACE were performed on the Marathon library using methods as described by Clontech. The 5′ gene specific primer sequence was AGAGAAGGGAAGGAGGGAAG (hUCP2.CDSR3). The 3′ gene specific primer sequence was CATCTCCTGGGACGTAGC (hUCP2.CDSF3). PCR conditions were 94° C. 30 sec, 64° C. 30 sec, 72° C. 1 min for 30 cycles on an MJ Research PTC-200, with Clontech KlenTaq Plus DNA polymerase. F. Oligonucleotides that can be used to amplify the UCP2 coding sequence. Names are: hUCP2.CDS (F or R, pair number) where hUCP2 means human UCP2, CDS means coding sequence and F or R refers to forward or reverse primer. PCR conditions are in an MJ research PTC-200 96-well machine. Clontech KlenTaq plus DNA polymerase were used: 94° C. 30 sec, 60° C. 30 sec, and 68° C. 2 min. Times 30 cycles.

FIG. 2. Flow cytometry analysis of membrane potential following the expression of UCP2 in S. cerevisiae; comparison of wild-type UCP1 and non-active UCP1 mutants. The number of cells (counts, Y-axis) is represented on a logarithmic scale of 1024 channels. The X-axis is a logarithmic scale of fluorescence intensity. A shift of the curve towards the left indicates a decreased mitochondrial potential. The horizontal bar on the left indicates the position of cells treated with mCICCP, a chemical uncoupler. The expression of UCP2, UCP1 or UCPmut mRNA in recombinant yeast, as well as its absence in wild type yeast, was checked by Northern blots.

FIGS. 3A-C. A. Tissue distributions of UCP2 in humans and mice. Human UCP2 mRNA was detected on a human multiple tissue Northern blot containing 2 μg mRNA per lane (Clontech, Palo Alto). This blot was probed with ³²P-labeled human UCP2 insert from IMAGE clone 129216. The 32P-labeled insert was used to probe the Master blot in ExpressHyb solution (Clontech, Palo Alto, Calif.). Expression of UCP1 and UCP2 in mice was examined in various tissues of mice maintained at 23° C. 20 μg RNA was loaded in each lane except the WAT lane which has 4 μg. UCP1 mRNA size is 1.5 kb, while UCP2 mRNA size is 1.6 kb. The UCP1 probe is the HSU 28480 clone, while UCP2 clone is GenBank accession number U69135 (DR) . Abbreviations: H, heart; B, brain; Pl, placenta; Lu, lung; L, liver; M, skeletal muscle; K, kidney; P, pancreas; Wat, white adipose tissue; Bat, brown adipose tissue; and M1 and M2 are thigh and abdominal muscle, respectively.

B. Chromosomal location of Ucp2 on distal mouse chromosome 7. The approximate position in centi-Morgan (cM) distance from the centromere, relationship among other genes and corresponding human chromosomal positions of orthologous genes are shown. The Ucp2 gene segregated in all 114 meiotic events examined with β-arrestin (Arrb1), a gene previously mapped to this position on both mouse chromosome 7 and to the long arm of human chromosome 11 (band q13). The other symbols and corresponding names are: Tyr, tyrosinase; D7Was12, DNA segment Chr 7, Washington 12; Omp, olfactory marker protein; Hbb, hemoglobin beta chain complex; tub, tubby mutation; Calc, calcitonin; and D7Mit40, DNA segment Chr 7 Mit 40.

C. Regulation of UCP2 mRNA levels in A/J and C57BL/6J by diet. 30 μg of epididymal fat pad white adipose tissue RNA from 2 mice from each strain and diet was electrophoresed and a Northern blot prepared. The high-fat diet is the same as described previously (Surwit et al, Diabetes 37:1163 (1988)). The blot was probed with 32P-labeled clone 129216 (human UCP2) and with cyclophilin. Amounts of UCP2 mRNA were determined with a Molecular Dynamics phosphorimager and were normalized with cyclophilin mRNA levels.

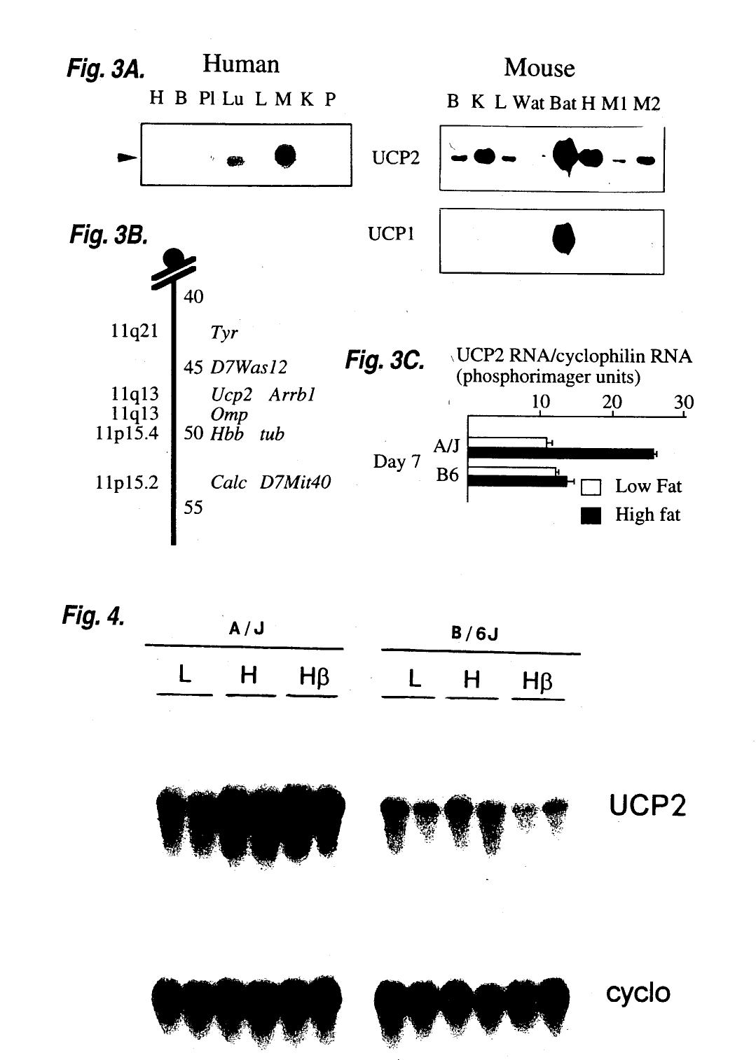

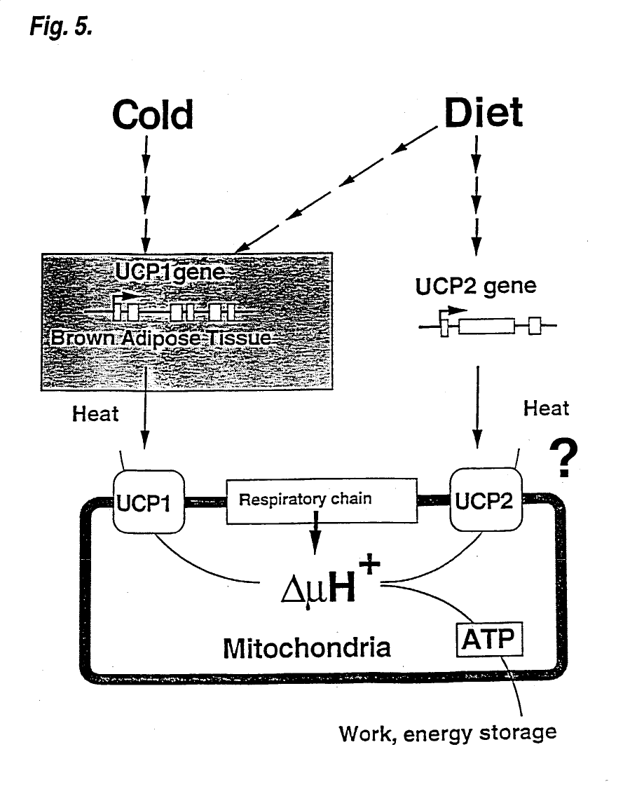

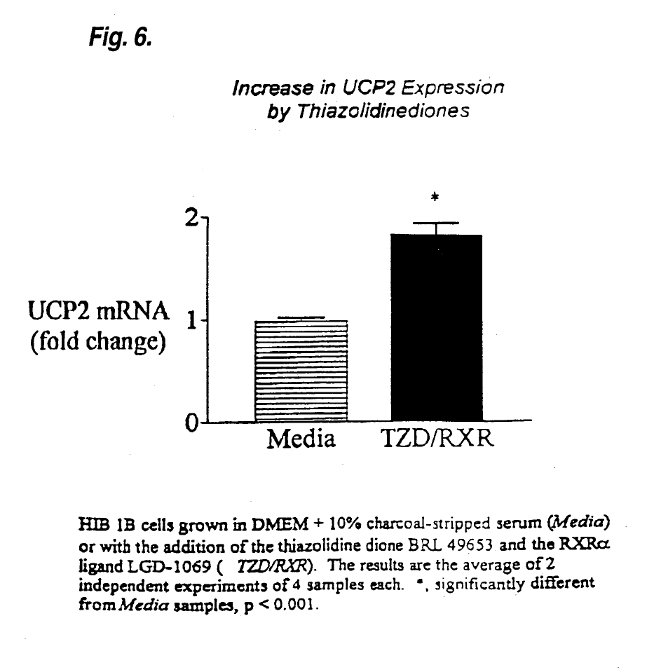

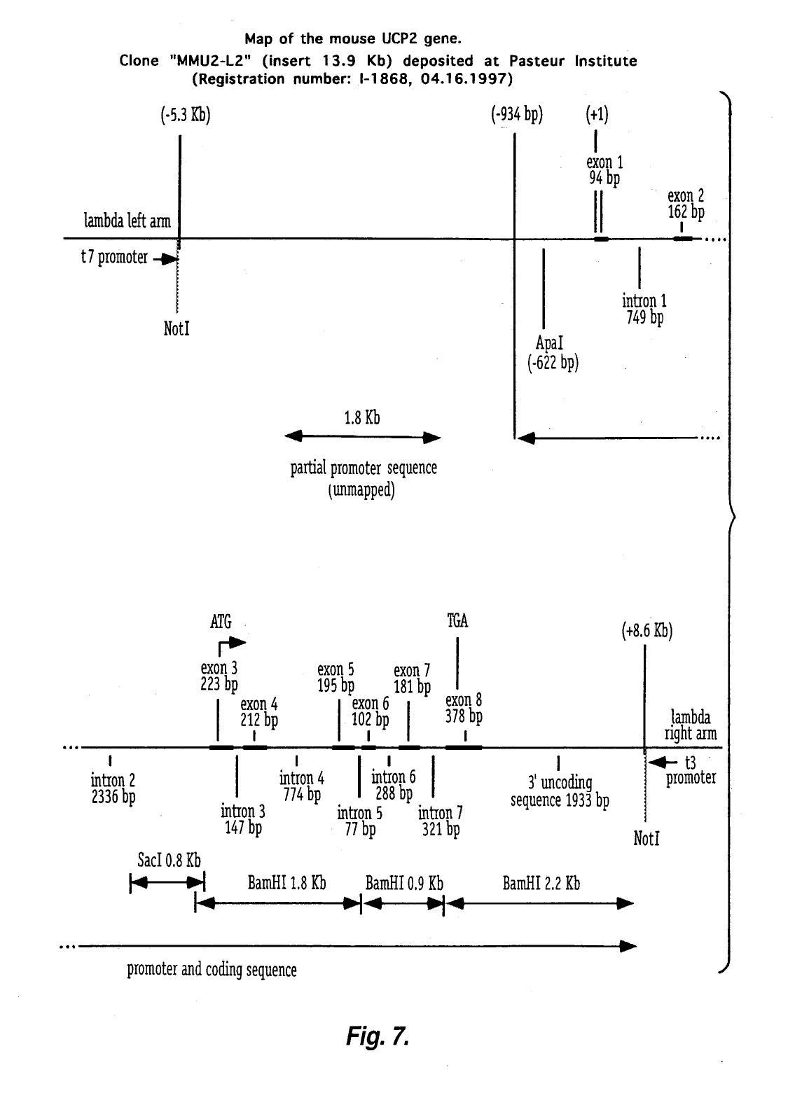

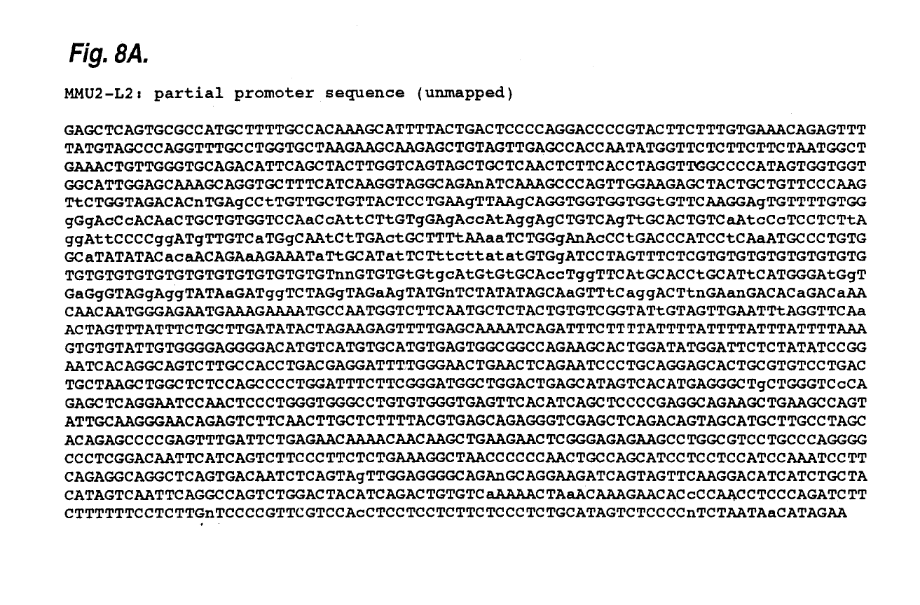

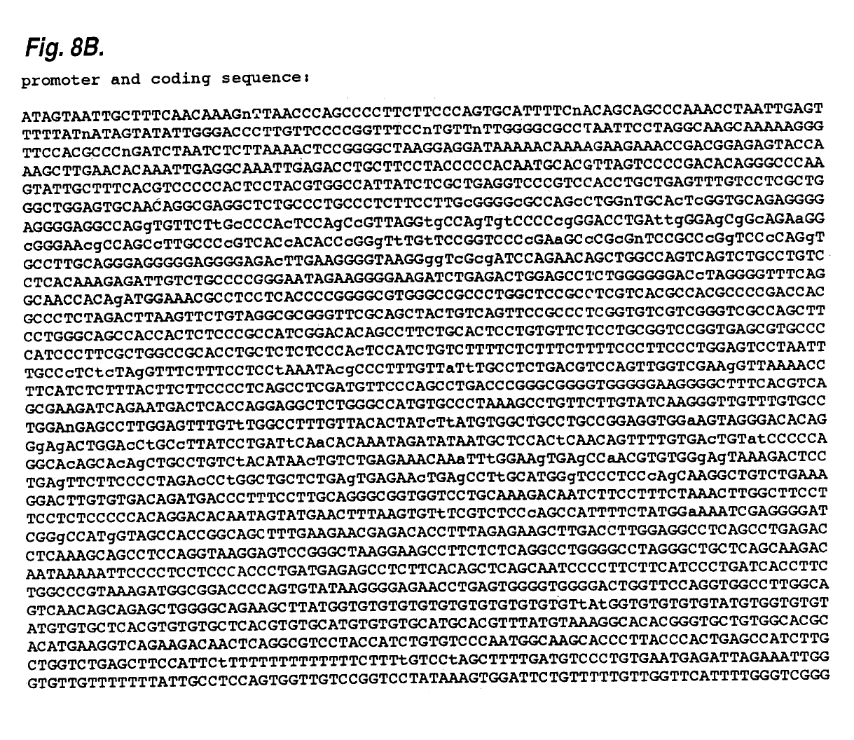

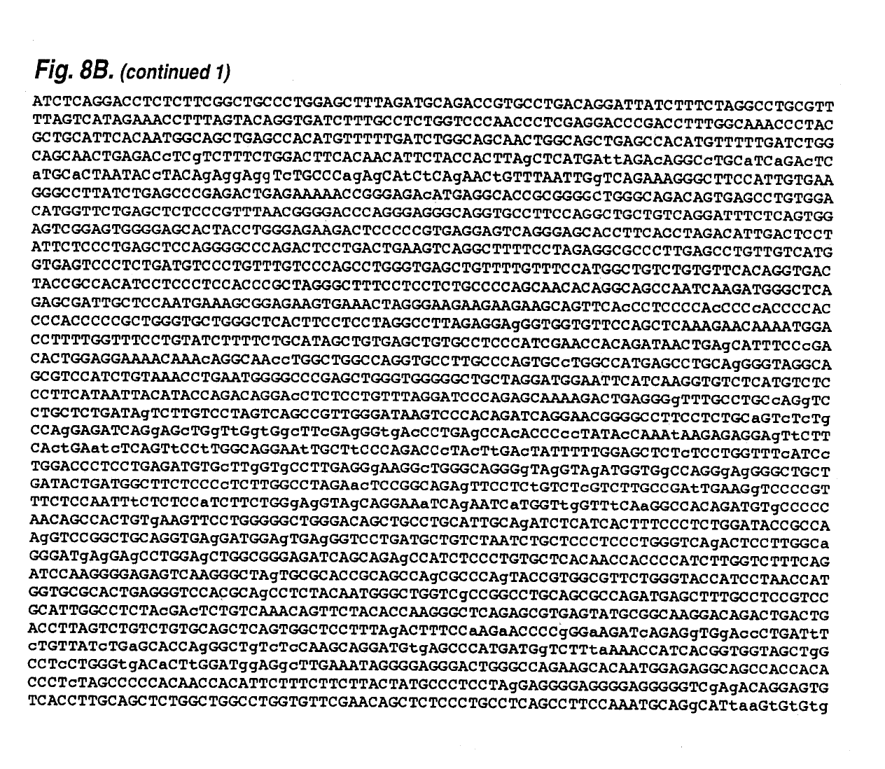

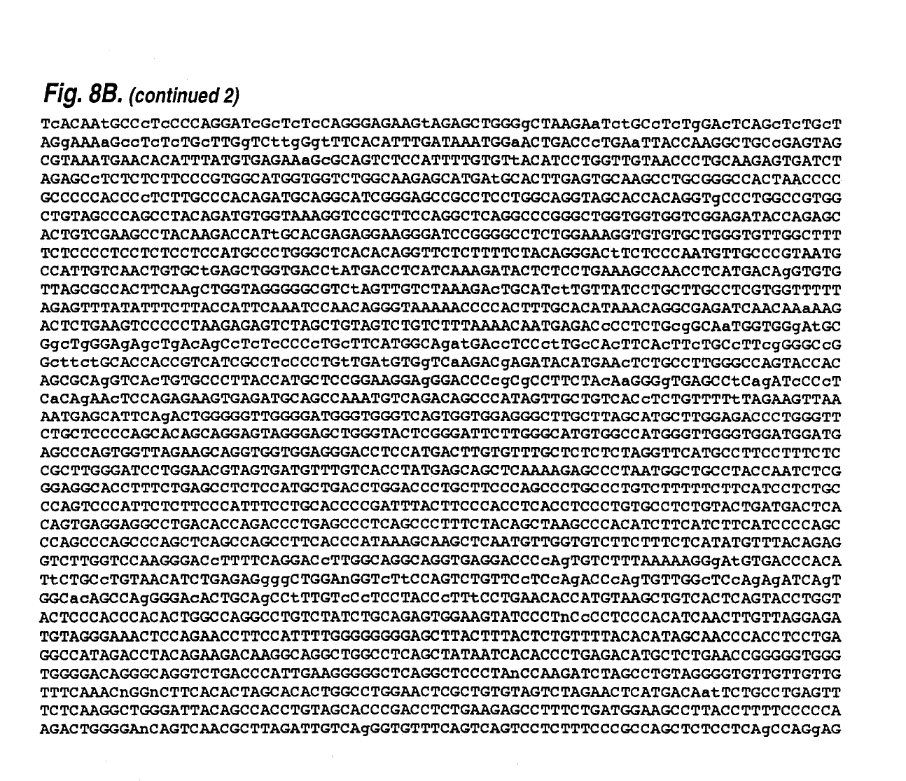

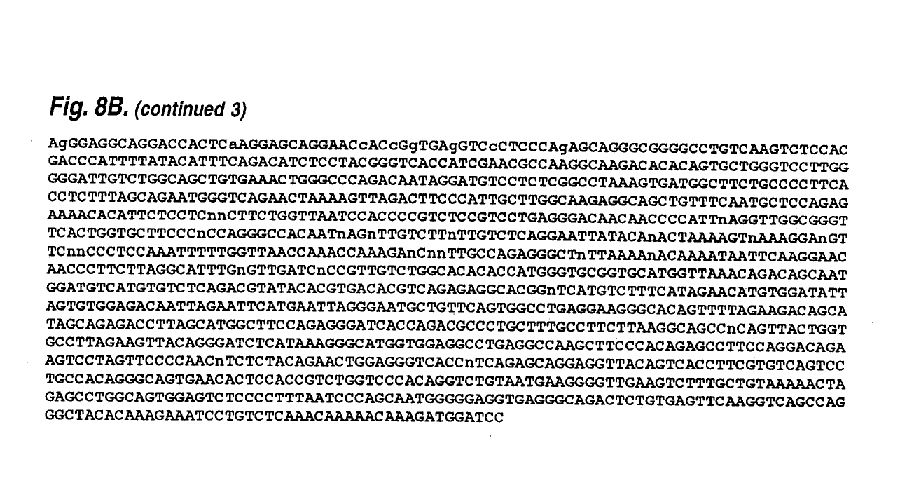

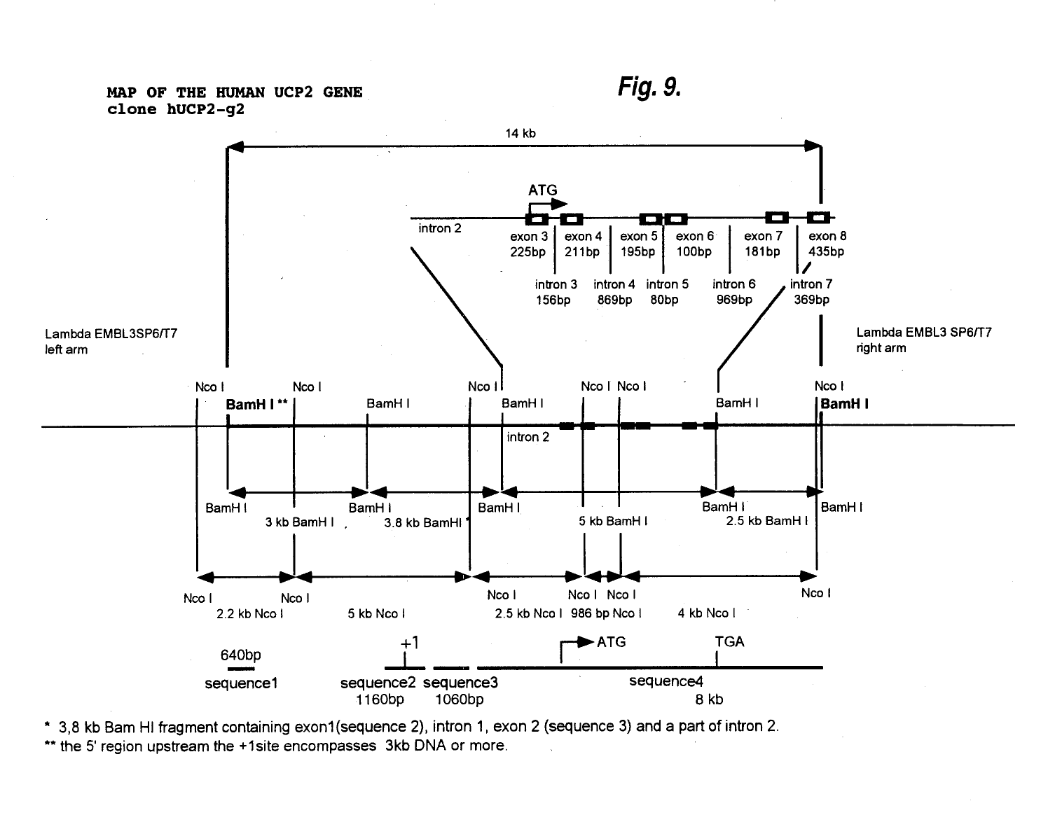

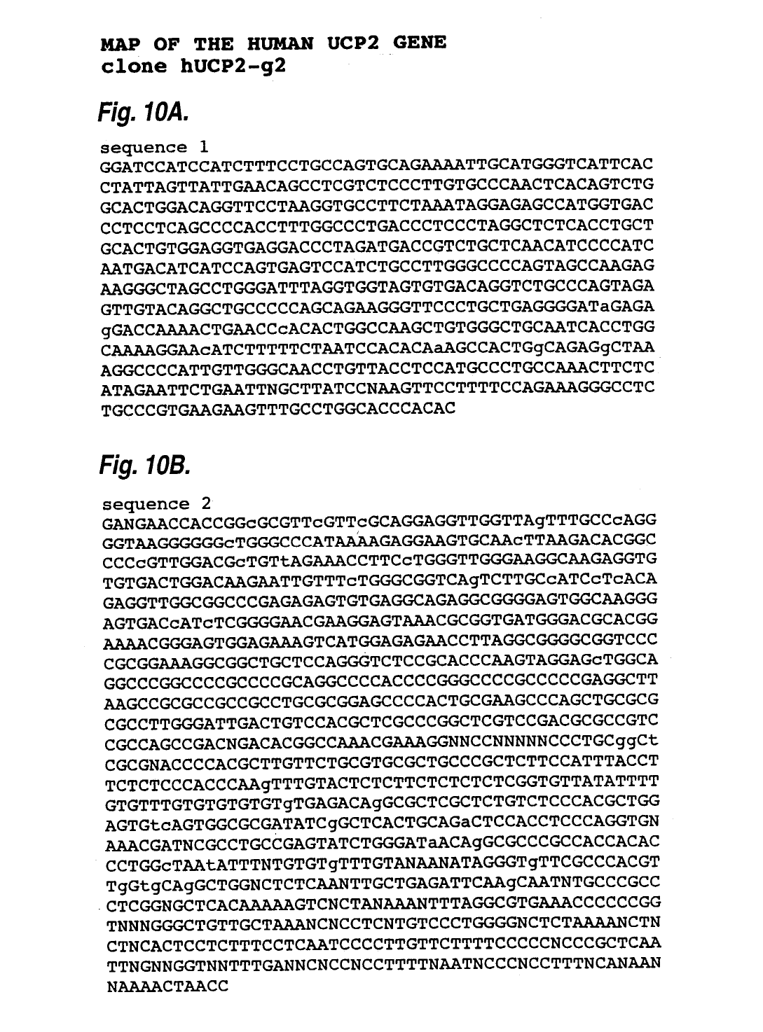

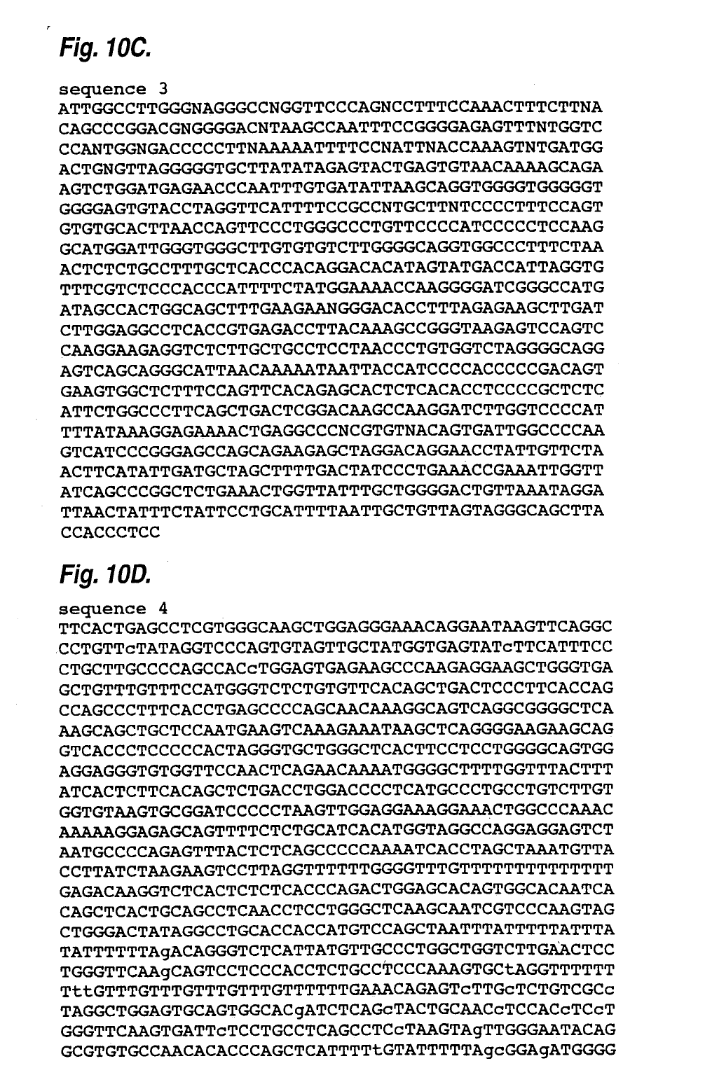

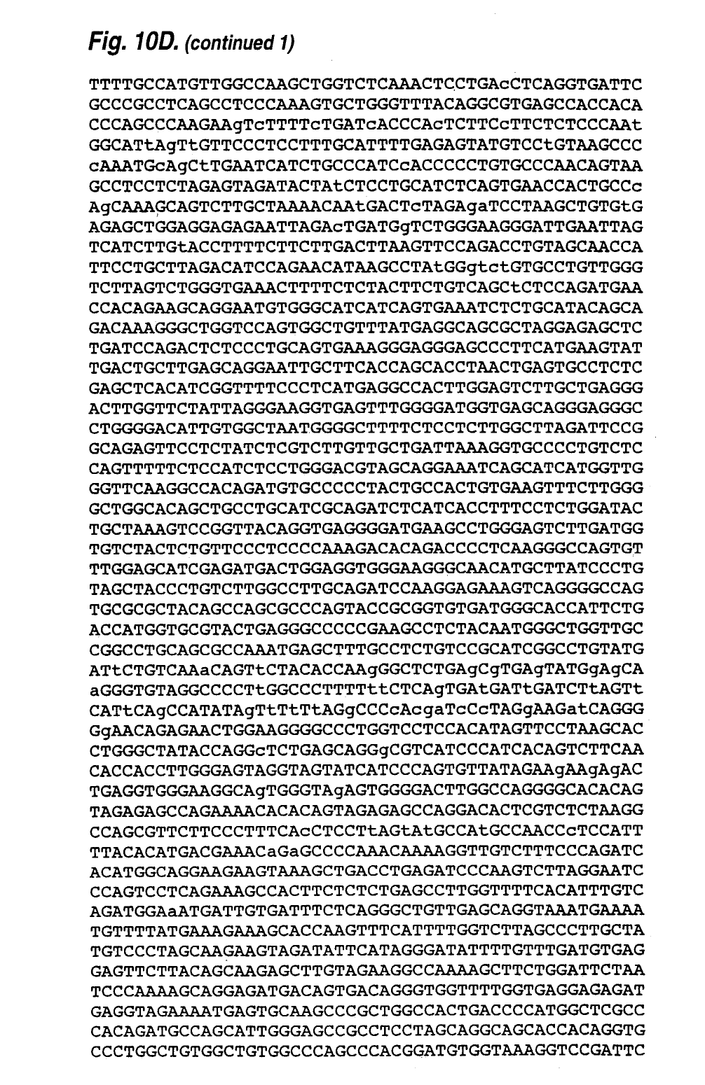

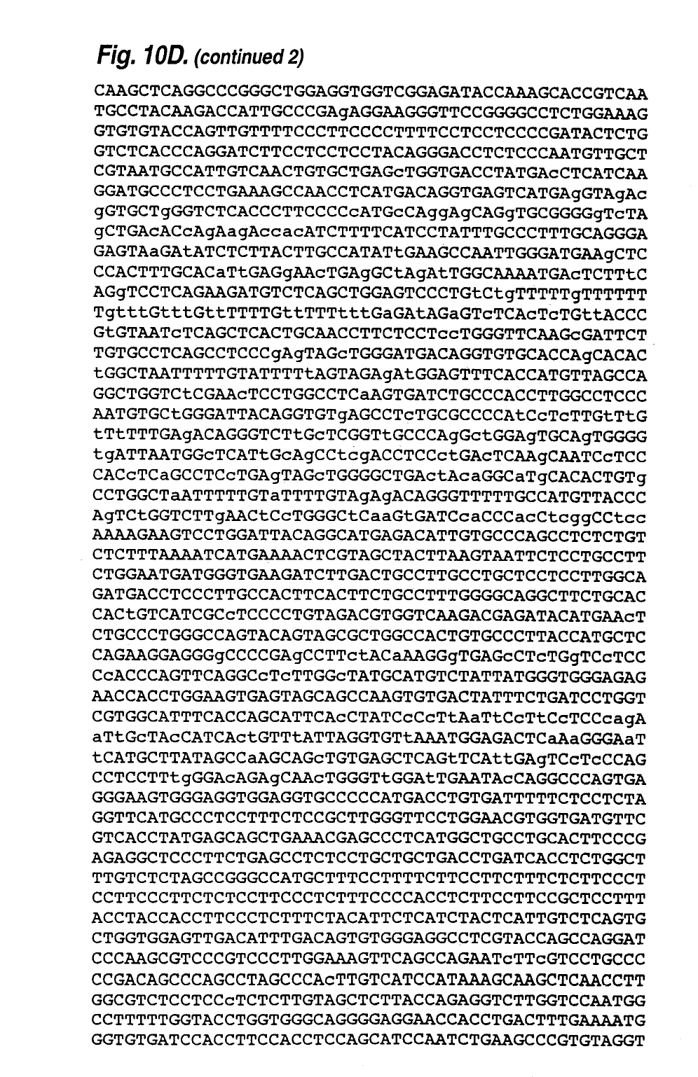

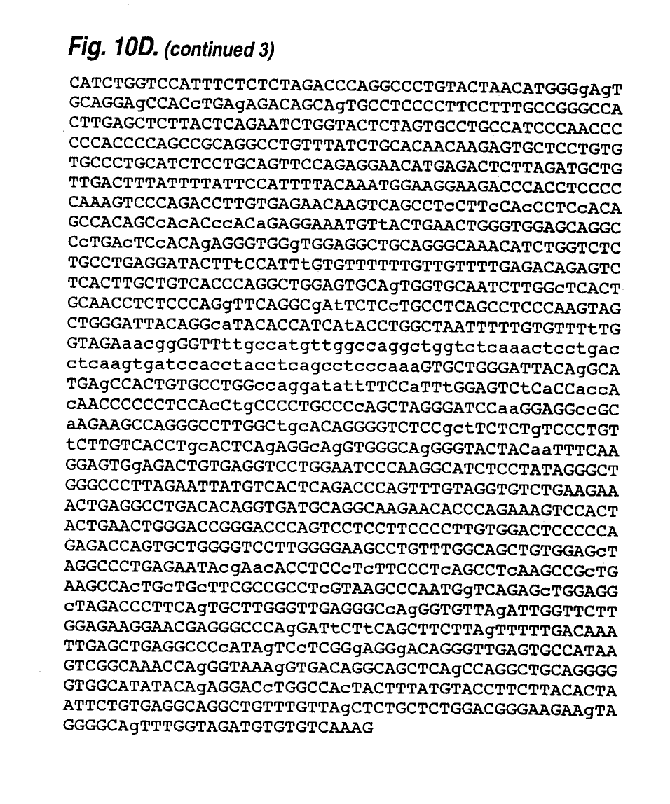

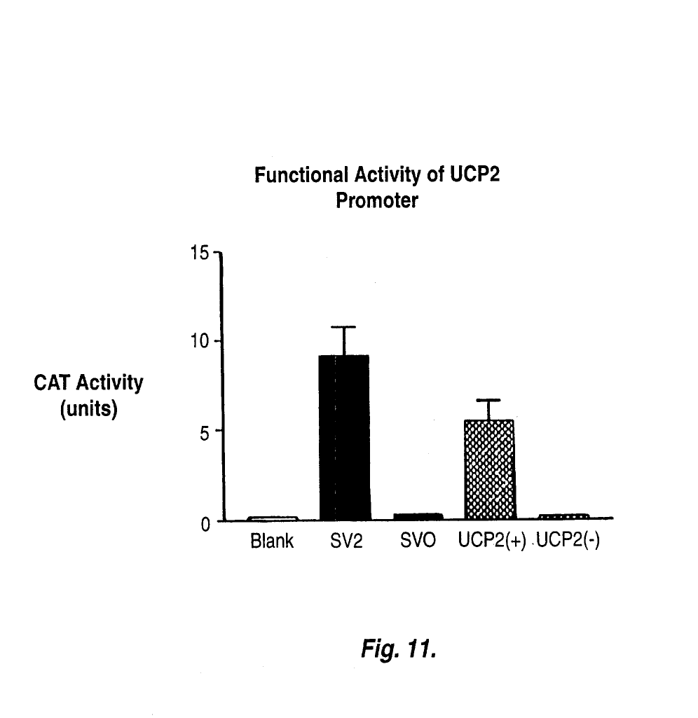

FIG. 4. Effect of diet on UCP2 mRNA levels in A/J and B6 mice. Methods are as described in FIG. 3C. Epididymal white adipose tissue was prepared from mice on a low fat diet (L), a high fat diet (Surwit et al, Diabetes 37:1163 (1988)) (H), or the high fat diet and the β3 adrenergic agonist CL316,243 (Hβ) for 25 days (Collins et al, Endocrin. 138:405 (1997)).

FIG. 5. Hypothesis about the regulation of resting metabolic rate and thermogenesis by UCP1 and UCP2. In mitochondria, the proton electrochemical gradient (ΔμH+) generated by the respiratory chain allows ATP synthesis by ATP synthase. In brown adipocyte mitochondria, uniquely, UCP1 short-circuits the proton circuit and oxidative energy is dissipated as heat in response to cold exposure. It has also been proposed that cafeteria diets may activate brown adipocyte thermogenesis (Rothwell et al, Nature 281:31 (1979)). In contrast, mitochondria of many tissues express UCP2, an homolog of UCP1, that may also lower the level of coupling of respiration to ADP phosphorylation. This may also lead to increased resting metabolic rate and body temperature and underlies “thermogenesis”. The present data show that UCP2 is not induced by cold but is induced by diet.

FIG. 6. Increase in UCP2 expression by thiazolidinediones.

FIG. 7. Map showing organization of the mouse UCP2 gene.

FIGS. 8A and B. Mouse UCP2 sequences. A. Partial promoter sequence. B. Promoter and coding sequence.

FIG. 9. Map showing organization of the human UCP2 gene.

FIGS. 10A-D. Sequences of four regions of the human UCP2 gene.

FIG. 11. Functional activity of UCP2 promoter. The following CAT reporter constructs were transfected into HIB-1B cells: SV2 contains the SV40 T-antigen promter; SV0 is a promoter-less control construct; UCP2(+) and UCP2(−) indictes constructs containing the 246 bp fragment from the mouse UCP2 gene inserted in the “sense” orientation or the “antisense” orientation, respectively. Twenty-four hours following their introduction into HIB-1B, the cells were harvested, a soluble extract of the cells was prepared and the level of CAT activity was measured by the TLC method. The data shown are the mean ± sd of two determinations, and the CAT activity is determined as acetylated chloramphenicol product/total, and is expressed as an artibrary unit. The sample marked as Blank is the unreacted chloramphenicol substrate.

### SUMMARY OF THE INVENTION

The present invention relates, in general, to a protein that can effect partial uncoupling of respiration. The protein, designated uncoupling protein-2 (UCP2), is linked to hyperinsulinemia, resting metabolic rate, glucose intolerance, diabetes, obesity, anorexia, cachexia and syndrome X (Reaven, Diabetes 37:1595 (1988); De Franzo et al, Diabetes Care 14:173 (1991)). The invention also relates to nucleic acid sequences encoding UCP2 and to diagnostic methodologies based, for example, on a determination of levels of UCP2 expression. Further, the invention relates to therapies involving modulating UCP2 expression and/or activity. In addition, the present invention relates to methods of screening compounds for their suitability for use in such therapies.

Objects and advantages of the present invention will be clear from the description that follows.

### DETAILED DESCRIPTION OF THE INVENTION

The present invention relates to a protein that is involved in energy balance, body weight regulation and thermoregulation. The protein, UCP2, is widely expressed in mammalian tissues, including human tissues, and UCP2 mRNA levels in white fat are elevated in response to fat feeding. The UCP2 gene sequence maps to a chromosomal region linked to obesity and hyperinsulinemia.

The identification of UCP2 provides an explanation for a proton leak that has been reported in mitochrondria (Brand et al, Biochem. J. 275:81 (1991); Porter et al, Am. J. Physiol. 269:R1213 (1995); Porter et al, Nature 362:628 (1993)). This leak is related to the standard metabolic rate which varies inversely with body mass of various species and with hormonal status (Brand et al, Bio. Chem. J. 275:81 (1991); Porter et al, Nature 362:628 (1993); Rice et al, Obesity Research 4:441 (1996)). Thus, UCP2 proton conductants can be expected to vary with membrane potential, as is the case with UCP1 (Diolez et al, in 8th European Bioenergetic Conference, Valencia, Spain (1994)). This property permits the leak to operate without collapsing membrane potential.

In brown adipose tissue, UCP1 creates a pathway that allows dissipation of the electrochemical gradient of protons across the inner membrane, without coupling to any other energy consuming process (Nedergard et al, in Molecular Mechanisms and Bioenergetics 23:385-420, ed. Ernster, L., Elsevier Science (1992)). The consequences are lower efficiency of oxidative phosphorylation and increased heat production. Previously, this was expected to be a general property of the membrane (involving the phospholipid composition) or a side effect of a known protein such as the ADP/ATP carrier. As shown in FIG. 5, it is now known that UCP2, a thermogenic and resting metabolic rate protein that is directly regulated by diet, underlies this phenomena. Further, the expression of UCP2 in spleen, bone marrow, macrophages and lymphocytes indicates that this protein also underlies thermogenic responses (e.g. fever) to inflammatory stimuli.

The present invention relates generally to a nucleic acid sequence encoding UCP2, particularly, a mammalian UCP2, more particularly, human UCP2, or portion of that encoding sequence. The invention further relates to the encoded protein, polypeptide or peptide. The term “portion”, as used herein, and as applied to nucleic acid sequences, refers to fragments of at least 15 or 30 bases, preferably, at least 50 bases, more preferably, at least 100 bases and, most preferably, at least 150, 300 or 500 bases. As applied to proteins, the term “portion” relates to peptides and polypeptides of at least 5 or 10 amino acids, preferably, at least 17 amino acids, more preferably, at least 33 amino acids and most preferably, at least 50, 100 or 240 amino acids. The invention also relates recombinant molecules comprising the above nucleic acid sequences and to host cells transformed therewith. In addition, the invention relates to methods of making the protein, polypeptide or peptide encoded in the nucleic acid sequence by culturing the transformed host cells under appropriate conditions. Furthermore, the invention relates to methods of screening compounds for the ability to bind to or alter the activity of or the expression of the UCP2 gene product. In addition, the invention relates to diagnostic and treatment methodologies based on UCP2 and its encoding sequence.

### UCP2 Encoding Sequence and Methods of UCP2 Production Using Same, UCP2 and Anti-UCP2 Antibodies

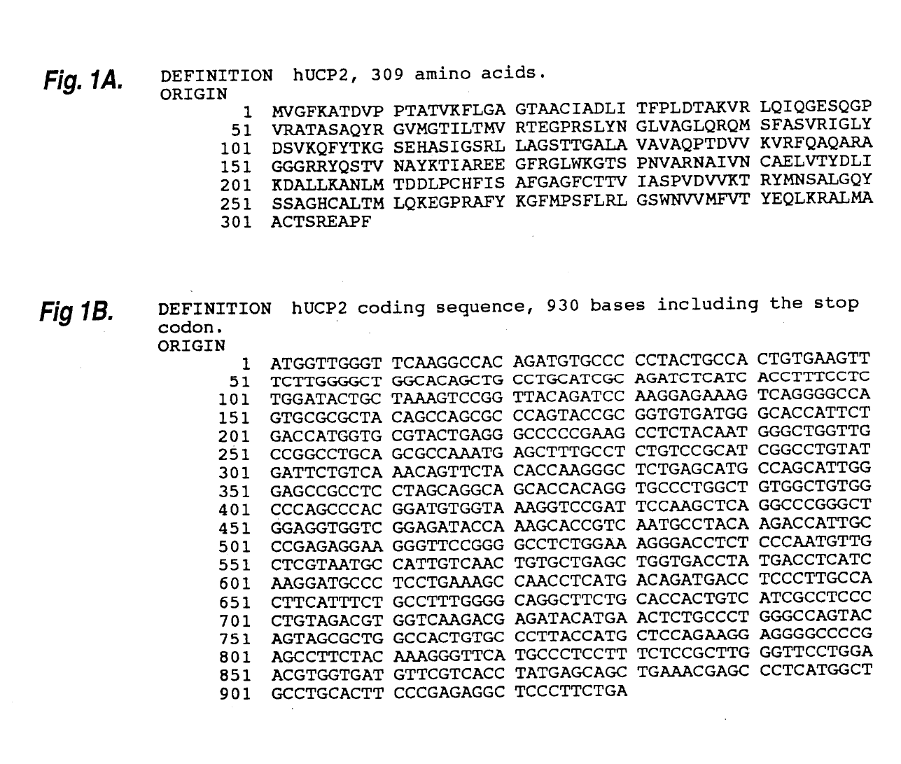

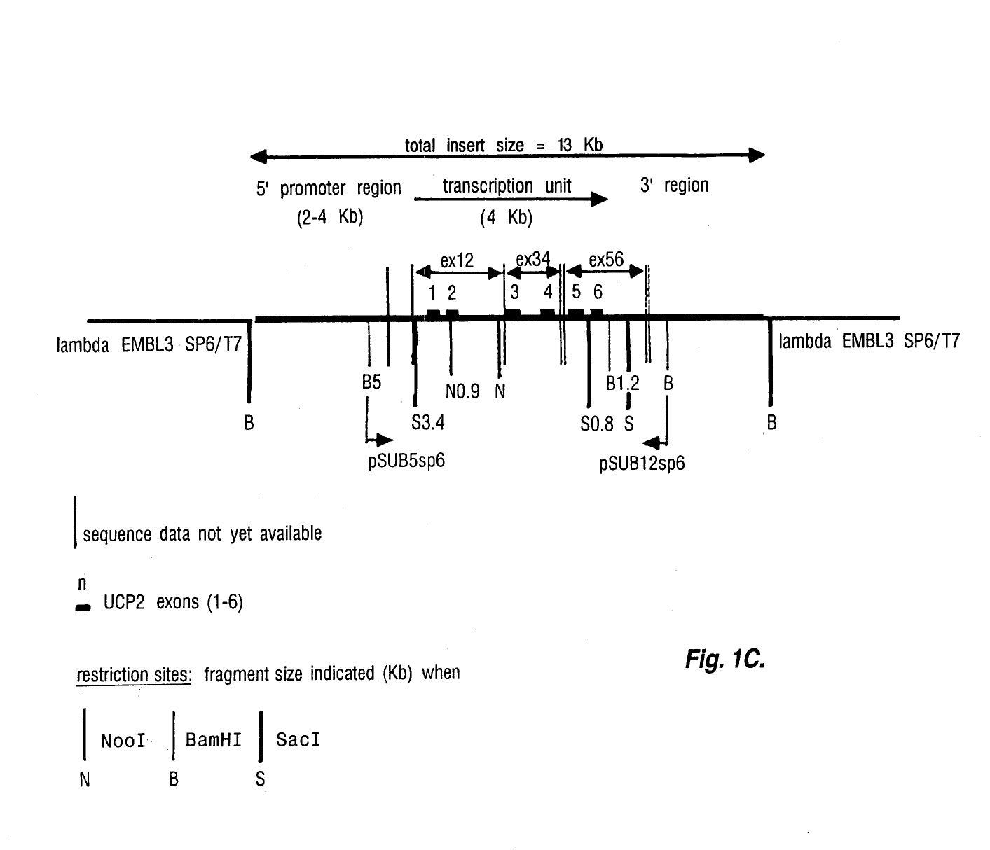

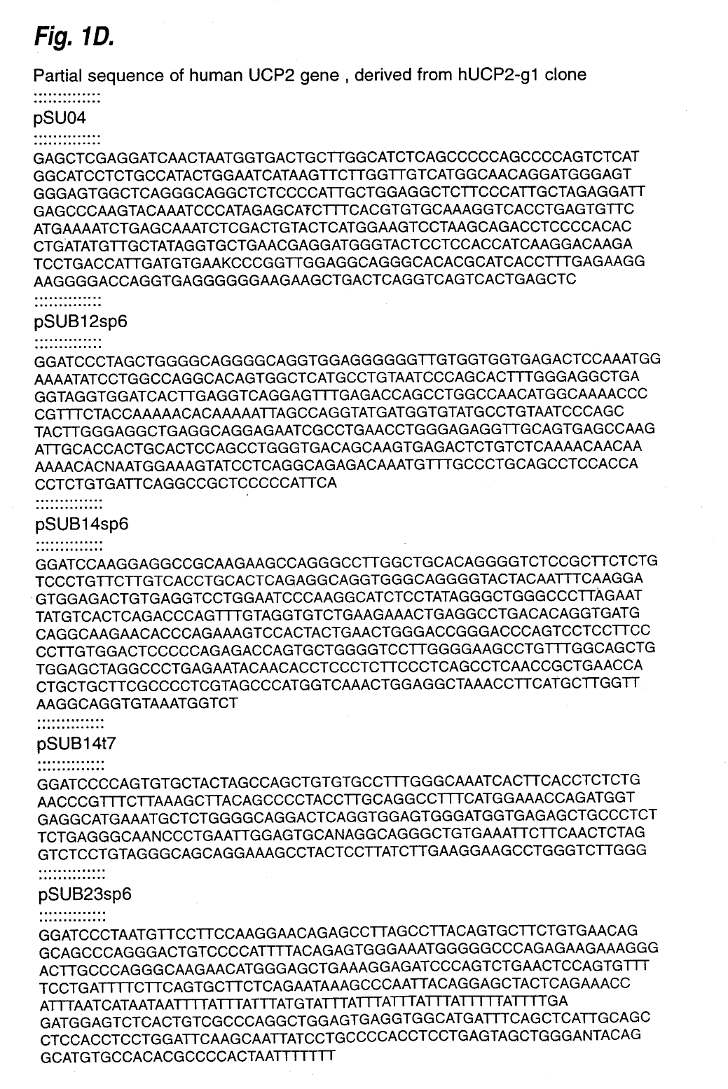

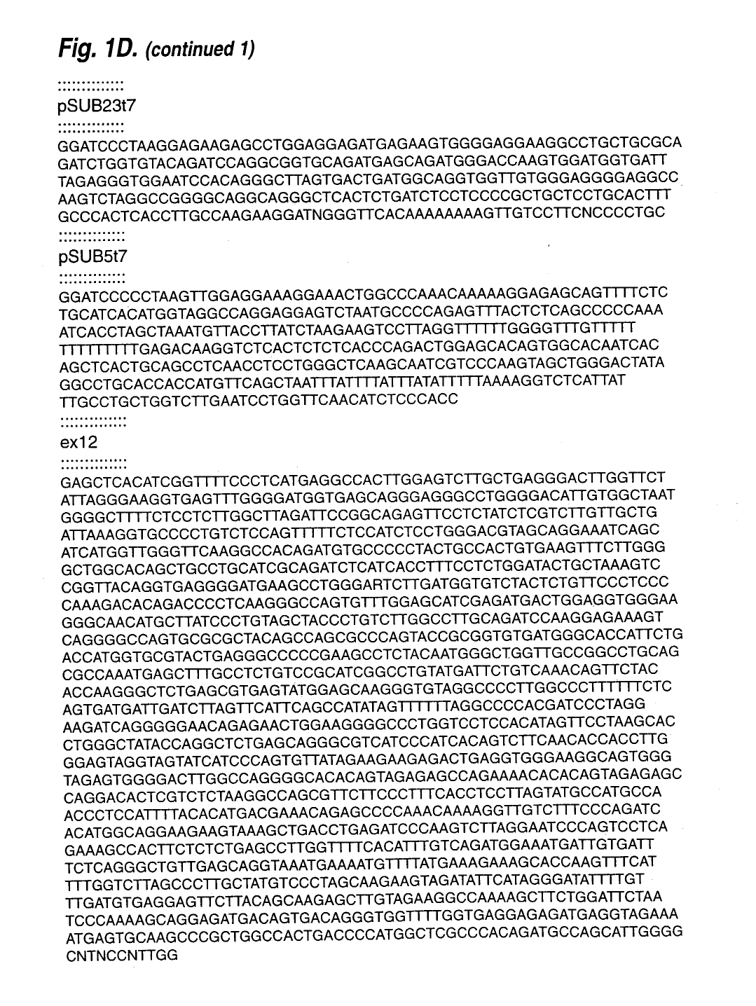

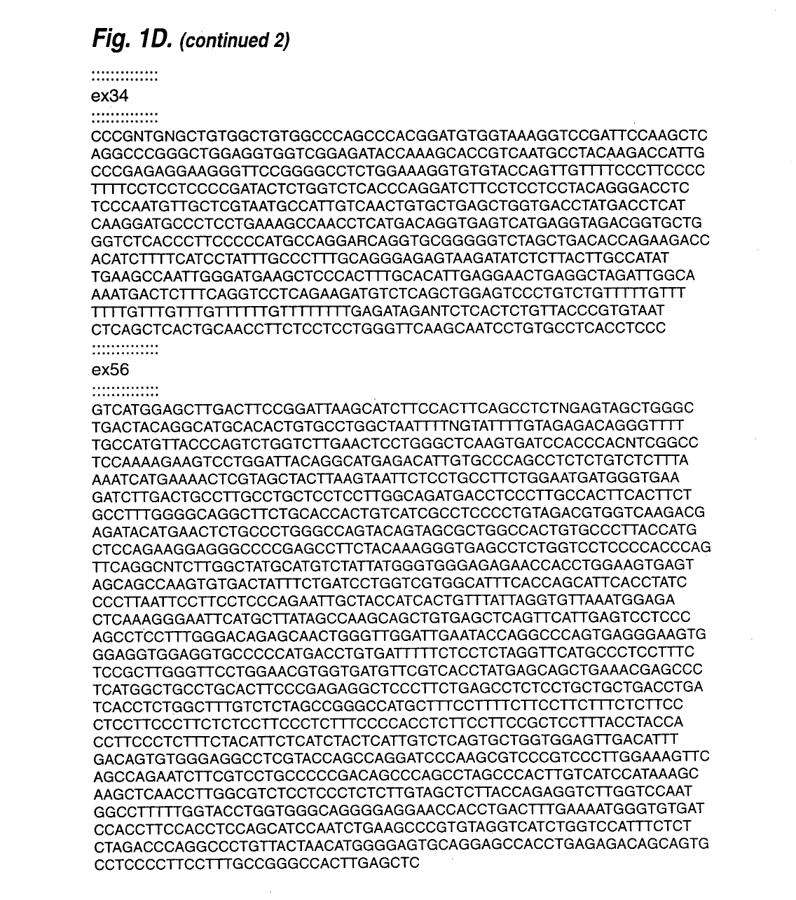

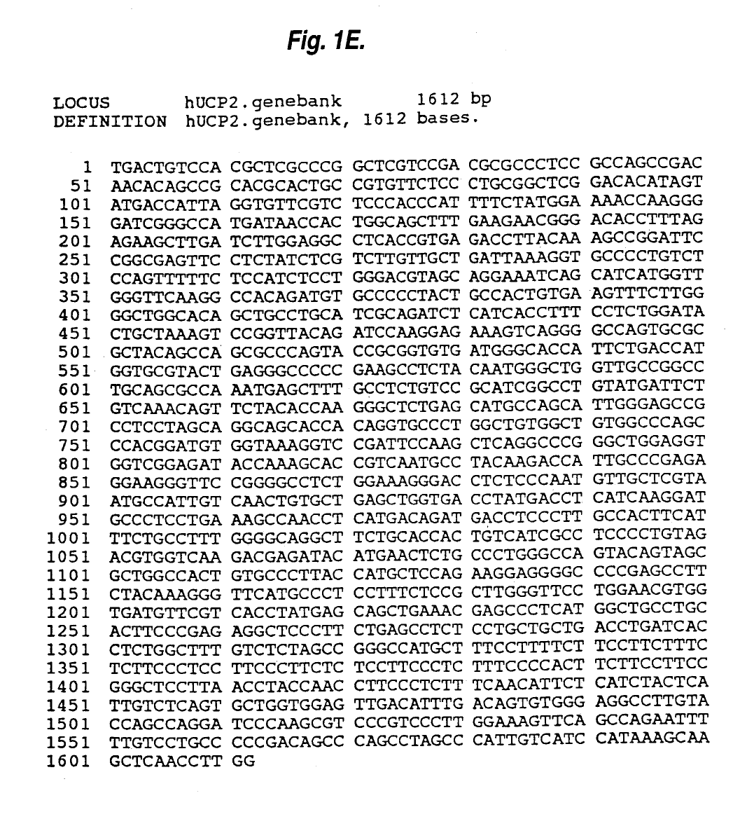

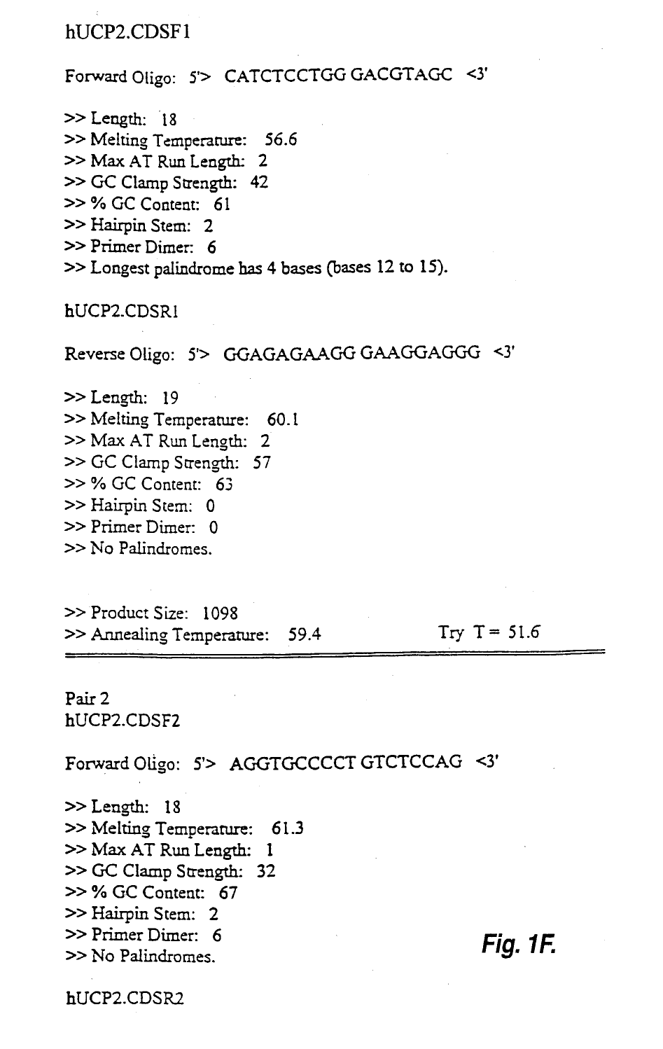

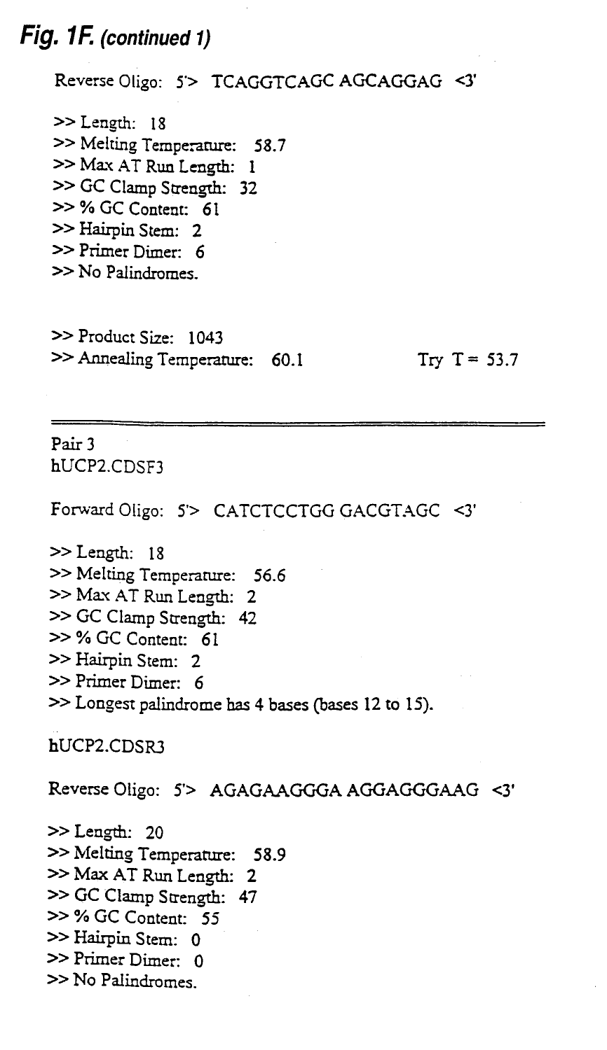

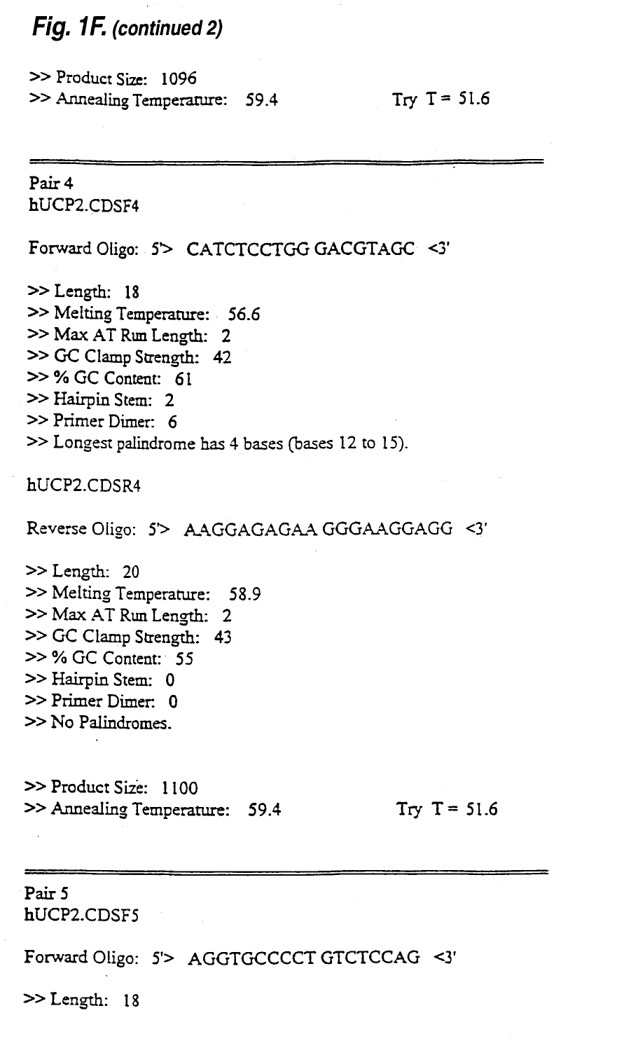

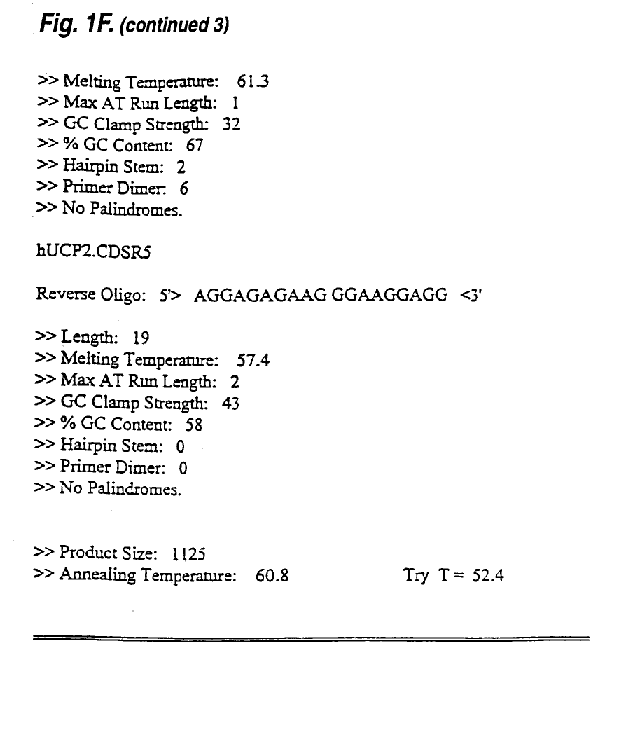

The present invention relates to nucleotide sequences that encode a protein that partially uncouples respiration (as does UCP1) in non brown (as well as brown) adipose tissue, for example, mammalian UCP2, particularly, human UCP2, or portions thereof as defined above (examples of such portions include sequences encoding the 10 N-terminal amino acids, and sequences encoding the mitochondrial carrier protein motifs of FIG. 1A). In particular, the present invention relates to nucleotide sequences that encode the amino acid sequence given in FIG. 1A, or portions thereof as defined above (the specific DNA sequence encoding UCP2 given in FIG. 1B being only an example). Further, nucleotide sequences to which the invention relates include those encoding substantially the same protein as shown in FIG. 1A, for example, inter- and intra-species variations thereof (see Example IX), as well as functional equivalents of the amino acid sequence shown in FIG. 1A. The invention further relates to nucleotide sequences substantially identical to the sequence shown in FIG. 1B. A “substantially identical” sequence is one the complement of which hybridizes to the nucleic acid sequence of FIG. 1B at 42° C. in 50% formamide, 1×saline/sodium citrate (SSC) containing 0.25% SDS and which remains bound when subjected to washing at 52-55° C. with 0.1-0.2×SSC containing 0.1% SDS (note: 20×SSC=3 M sodium chloride/0.3 M sodium citrate). The invention also relates to nucleic acids complementary to those described above.

The present invention also relates to a recombinant molecule comprising a nucleotide sequence as described above and to a host cell transformed therewith. Using standard methodologies, well known in the art, a recombinant molecule comprising a vector and a nucleotide sequence encoding the UCP2 protein, or portion thereof as defined above, can be constructed. Vectors suitable for use in the present invention include plasmid and viral vectors. Plasmid vectors into which a nucleic sequence encoding the UCP2 protein, or portion thereof, can be cloned include any vectors compatible with transformation into a selected host cell. Such vectors include vectors suitable for introduction into yeast and insect cells, generally, mammalian expression vectors suitable for expression in host cells, which vectors can include sequence elements that enhance transcription and/or prolong mRNA halflife in the cell (e.g. β-globin gene 3′ untranslated region) specifically, pUC-based E. coli vectors, pYeDPUCP2, pSelectUCP2, and PECE-UCP2. The nucleotide sequence of the invention can be present in the vector operably linked to regulatory elements, for example, a promoter. Suitable promoters include, but are not limited to, tissue specific promoters (e.g. leptin gene promoter or aP2 gene promoter specific for adipose cells, muscle creatine kinase promoter specific for skeletal muscle and lymphoid cell promoters), muscle actin promoter, interleukin promoter, cMV, SV40 and MMTV promoters.

As indicated above, the recombinant molecule of the invention can be constructed so as to be suitable for transforming a host cell. Suitable host cells include prokaryotic cells, such as bacteria, lower eukaryotic cells, such as yeast, and higher eukaryotic cells, such as mammalian cells, and insect cells. The recombinant molecule of the invention can be introduced into appropriate host cells by one skilled in the art using a variety of known methods.

The present invention further relates to a method of producing UCP2, or portions thereof as defined above. The method comprises culturing the above-described transformed host cells under conditions such that the encoding sequence is expressed and the protein thereby produced.

In addition to the nucleic acids described above, the present invention also relates to UCP2 gene sequences, including introns, exons and flanking regions (e.g. the UCP2 promoter), and to portions thereof suitable for use as probes or primers. The invention also relates to nucleic acid sequences corresponding to the entire expressed UCP2 sequence (e.g. UCP2 mRNA or corresponding cDNA), as well as portions thereof suitable, for example, for use as probes or primers.

A human UCP2 genomic clone, hUCP2-g1, was deposited on Jan. 13, 1997, and was given Accession No. I-1806. A further human genomic clone (designated hUCP2-g2 and a mouse genomic clone (designated MMU2-L2) were deposited at the Collection Nationale de Cultures de Microorganismes, Institut Pasteur, 25, Rue du Docteur Roux, 75724, Paris CEDEX 15, France, under the terms of the Budapest Treaty, on Apr. 16, 1997, and were given Accession Nos. I-1867 and I-1868, respectively. hUCP2-g2 was cloned from the same library used to clone hUCP2-g1. A 500 bp DNA corresponding to the 5′ end of hUCP2-g1 was used to screen the genomic library. MMU2-L2 was cloned from a mouse genomic library screened using the mouse UCP2 cDNA. The genomic library was from Strategene (La Jolla, C: 129SVJ Mouse genomic library—catalog number 946306—in the Lamda FIX II vector; the amplification host was XL1-blue MRA(P2). Sequence 2 of hUCP2-g2 is 1161 bp long and goes from position −511 to +650. Therefore, this fragment contains the putative human promoter region. A map of the human UCP2 gene is shown in FIG. 1C and the sequences of several subcloned fragments are given in FIG. 1D. (See also FIGS. 9 and 10.) Two further deposits (hUCP2-5′ and hUCP2-3′) were made at the American Type Culture Collection, 12301 Parklawn Drive, Rockville, Md. 20852, USA, under the terms of the Budapest Treaty, on Jan. 10, 1997. These deposits were given Accession Nos. 97850 and 97849, respectively. These deposits comprise plasmids containing inserts from normal human lung cDNA. The plasmids are in the Invitrogen pCR2.1 vector. hUCP2-5′ includes sequence from base pair (bp) 1 up to bp 1375, thus including the entire 5′ untranslated sequence and the entire coding sequence. hUCP2-3′ includes sequence from bp 313 up to bp 1612, thus including the entire coding sequence and the entire 3′ untranslated region. A bacterial artificial chromosome (BAC) clone for human genomic UCP2 DNA has been isolated. This clone, hUCP2.BAC, was deposited with the American Type Culture Collection, 12301 Parklawn Drive, Rockville, Md. 20852, USA, under the terms of the Budapest Treaty on Apr. 18, 1997. The clone was isolated by hybridizing hUCP2 probe to the human BAC library prepared by Genome Systems. The probe was PCR product for human UCP2 produced from human lung cDNA and amplified with hUCP2.cds3 primers as described in Example IX. BAC clone inserts range from 90 to 300 kb in length, with an average of 120 kb. Hybridization and isolation of hUCP2.BAC were performed by Genome Systems using their standard techniques. Since the human UCP2 gene is approximately 20 kb, hUCP2.BAC is believed to contain the entire gene as well as the entire promoter. hUCP2.BAC has also been digested with EcoRI and subcloned into the pZERO vector of Invitrogen. These subclones have been hybridized to hUCP2-5′ probe to identify clones with the human promoter. They have also been hybridized to a (CA)14 oligo to identify a polymorphic CA repeat. Consensus sequence for the entire human expressed UCP2 is shown in FIG. 1E.

Nucleic acid sequence(s) of the invention can be used, in accordance with standard protocols, as probes and primers. Oligonucleotides suitable for amplifying the human UCP2 coding sequence are given in FIG. 1F (pairs 1 and 3 exhibit little non-specific amplification).

The present invention further relates to mammalian UCP2, particularly, human UCP2, substantially free of proteins with which it is normally associated, or portions thereof as defined above. The proteins, polypeptides and peptides of the invention can be produced recombinantly using the nucleic acid sequences as described above, or chemically using known methods. The protein of the invention can be produced alone or as a fusion product, for example, with a protein such as green fluorescent protein, MaIE protein, glutathione S transferase, glutathione, thrombin, and polyhistidine. Such fusion products can be produced recombinantly. For example, the coding sequence of the invention (e.g. the sequence encoding human UCP2) can be cloned in frame with a sequence encoding another protein (see above examples) the fusion product expressed in an appropriate host cell.

The proteins, polypeptides and peptides of the invention can be used as antigens to generate UCP2 specific antibodies. Methods of antibody generation are well known in the art. Both monoclonal and polyclonal antibodies are included within the scope of the invention, as are binding fragments thereof. One skilled in the art will appreciate that such antibodies can be used to selectively identify and isolate UCP2 and portions thereof. In addition, the antibodies can be used to block activity of UCP2.

### Compound Screens

The present invention also relates to methods of screening compounds for their ability to modulate (e.g. increase or inhibit) the activity or expression of UCP2. More specifically, the present invention relates to methods of testing compounds for their ability either to increase or to decrease expression or activity of UCP2. The assays are performed in vitro or in vivo. In vitro, cells expressing UCP2 are incubated in the presence and absence of the test compound. By determining the level of UCP2 expression in the presence of the test compound (using, for example, Northern blots, immunoassays (e.g. RIA, Western blots or immunohistochemistry) or PCR), or the level of UCP2 activity in the presence of the test compound, compounds can be identified that suppress or enhance UCP2 expression or activity. Alternatively, constructs comprising the UCP2 promoter operably linked to a reporter gene (e.g. luciferase, chloramphenicol acetyl transferase, LacZ, green fluorescent protein, etc.) can be introduced into host cells and the effect of the test compounds on expression of the reporter gene detected.

Cells suitable for use in the foregoing assays include, but are not limited to, lymphoblasts, myocytes, adipocytes and hepatic cells, more specifically, C2C12 cells, 3T3 cells of adipocyte lineage, HIB-1B cells, rodent hepatoma cells, HepG2 cells, and B7 cells. (See Example V.)

Compounds that suppress or enhance UCP2 expression can also be identified using in vivo screens. In these assays, the test compound is administered (e.g. IV, IP, IM, orally, or otherwise), to the animal, for example, at a variety of dose levels. The effect of the compound on UCP2 expression is determined by comparing UCP2 levels, for example, in blood, muscle or fat tissue, using Northern blots, immunoassays, PCR, etc., as described above. Suitable test animals include rodents, primates, dogs and swine. Humanized mice can also be used as test animals, that is mice in which the endogenous mouse protein is ablated (knocked out) and the homologous human protein added back by standard transgenic approaches. Such mice express only the human form of a protein. Humanized mice expressing just the human UCP2 can be used to study in vivo responses of weight loss, fever, cachexia in response to potential agents regulating UCP2 protein or mRNA levels. As an example, transgenic mice have been produced carrying the human apoE4 gene. They are then bred with a mouse line that lacks endogenous apoE, to produce an animal model carrying human proteins believed to be instrumental in development of Alzheimers pathology. Such transgenic animals are useful for dissecting the biochemical and physiological steps of disease, and for development of therapies for disease intervention (Loring, et al, Neurobiol. Aging 17:173 (1996)).

Compounds that suppress or enhance UCP2 activity can be identified by contacting UCP2 with the test compound under conditions such that the compound can interact with (e.g. bind to) the protein. A system such as the yeast expression system described in Example I can be used. In such a system, the effect of the test compound on UCP2 activity can be determined, for example, by analyzing the alteration in membrane potential (e.g. using flow cytometry) (see Example I). (Comparable studies can be carried out in vivo by administering the test compound and measuring its effect on respiration and/or body temperature.)

In addition, using cellular systems such as the two-hybrid system (Fields et al, Nature 340:245 (1989); Chien et al, Proc. Natl. Acad. Sci. USA 88:94=578 (1991)), proteins can be identified that interact with UCP2.

Using in vivo (or in vitro) systems, it may be possible to identify compounds that exert a tissue specific effect, for example, that increase UCP2 expression or activity only in fat or muscle or cells of the immune system.

Screening procedures such as those described above are useful for identifying agents for their potential use in pharmacological intervention strategies. Agents that enhance UCP2 expression or activity can be used to treat disorders such as, hyperinsulinemia, glucose intolerance, diabetes, obesity and syndrome X. Compounds that suppress UCP2 expression or inhibit its activity can be used to treat wasting associated, for example, with cancer, AIDS, cachexia and anorexia. Agents that suppress UCP2 expression or inhibit its activity can also be used to induce hypothermia, for example, when advantageous in surgical settings, including transplantation. Such agents can also be used to block hyperthermia, for example, during thyroid storm. Compounds that enhance UCP2 expression or stimulate its activity can also be used to treat hypothermia. Given the similarity in the patterns of leptin and UCP2 expression, agents that modulate UCP2 can be expected to modulate leptin expression. However, leptin has been shown not to influence UCP2 expression in vitro or in vivo.

### Pharmaceutical Compositions

The present invention also relates to pharmaceutical compositions comprising, as active agent, the proteins, peptides, nucleic acids or antibodies of the invention. The invention also relates to compositions comprising, as active agent, compounds selected using the above-described screening protocols. Such compositions include the active agent in combination with a pharmaceutically acceptable carrier. In the case of naked DNA, the “carrier” can be gold particles. In the case of compositions to be applied topically, the composition can take the form of a gel, cream, ointment or lotion. The amount of active agent in the composition can vary with the agent, the patient and the effect sought. Likewise, the dosing regimen can vary depending on the composition and the disease/disorder to be treated.

### Detection/Diagnosis

The present invention further relates to methods of identifying individuals at increased risk for developing certain diseases/disorders, including hyperinsulinemia, glucose intolerance, type II diabetes, obesity, syndrome X, immunological dysfunction and body temperature dysfunction. One such method comprises: (a) obtaining from a mammal (e.g. a preobese human) a biological sample, (b) detecting the presence in the sample of a UCP2 gene product and (c) comparing the amount of the gene product present in the sample with that in a control sample. Advantageously, the biological sample is taken after the consumption of a high fat meal. In accordance with this method, the presence in the sample of altered (e.g. diminished) levels of UCP2 gene product indicates that the subject is predisposed to the above-indicated diseases/disorders.

Biological samples suitable for use in this method include biological fluids such as blood. Tissue samples (e.g. biopsies) can also be used in the method of the invention, including samples derived from muscle or fat. Cell cultures or cell extracts derived, for example, from tissue biopsies can also be used.

The detection step of the present method can be effected using standard protocols for protein/mRNA detection. Examples of suitable protocols include Northern blot analysis, immunoassays (e.g. RIA, Western blots, immunohistochemical analyses), and PCR.

The present invention also relates to methods of identifying individuals having elevated or reduced levels of UCP2, which individuals are likely to benefit from therapies designed to suppress or enhance UCP2 expression, respectively. As indicated above, a biological sample from a preobese subject can be screened for the presence of diminished levels of UCP2 gene product, particularly in response to high fat intake, the presence of depressed levels of the gene product, relative to a normal population (standard), being indicative of predisposition to obesity, type II diabetes or syndrome X. Such individuals would be candidates for anti-obesity therapy (e.g. treatment with appetite suppressants). The identification of elevated levels of UCP2 in a wasting patient (e.g. a cancer, AIDS or anorexia patient) would be indicative of an individual that would benefit from treatment with agents that suppress UCP2 expression or activity. The identification of low levels of UCP2 in a hypothermic patient or obese patient would be indicative of an individual that would benefit from agents that induce UCP2 expression or activity.

The present invention also relates to a kit that can be used in the detection of UCP2 expression products. The kit can comprise a compound that specifically binds UCP2 (e.g. binding proteins (e.g. antibodies or binding fragments thereof (e.g. F(ab′)₂ fragments)) or UCP2 mRNA (e.g. a complementary probe or primer), for example, disposed within a container means. The kit can further comprise ancillary reagents, including buffers and the like.

The diagnostic methodologies described herein are applicable to both humans and non-human mammals.

### Therapy

The present invention relates to methods of treating diseases/disorders such as hyperinsulinemia, glucose intolerance, diabetes, obesity, syndrome X, cancer and hypothermia by increasing UCP2 activity and/or expression. The invention also relates to methods of treating inflammation, anorexia and wasting (cachexia) (e.g. associated with cancer or AIDS), of reducing fever and blocking hyperthermia (e.g. thyroid storm) and to methods of inducing hypothermia (eg when advantageous for surgery and transplant), by decreasing UCP2 activity and or expression. These methodologies can be effected using compounds selected using screening protocols such as those described above and/or by using the gene therapy and antisense approaches described below.

Gene therapy can be used to effect targeted expression of UCP2, for example, in fat tissue and muscle to reduce fat depots or in cancer cells to cause thermodestruction or metabolic collapse/death of the cells. The UCP2 coding sequence can be cloned into an appropriate expression vector and targeted to a particular cell type(s) to achieve efficient, high level expression. Introduction of the UCP2 coding sequence into target cells can be achieved, for example, using particle mediated DNA delivery (William et al, Proc. Natl. Acad. Sci. USA 88:2726 (1991); Haynes, Advances in Drug Delivery Reviews 21:3 (1996)), direct injection of naked DNA (Levy et al, Gene Therapy 3:21 (1996)), liposomal DNA transport (Feigner, Human Gene Therapy 1:1791 (1996)), or viral vector mediated transport (Smith et al, Gene Therapy 3:190 (1996)). Tissue specific effects can be achieved, for example, in the case of virus mediated transport by using viral vectors that are tissue specific, or by the use of promoters that are tissue specific (e.g. leptin and aP2 promoters can be used to achieve expression in white adipose tissue and the myosin light chain kinase promoter can be used to achieve expression in skeletal muscle) (see also Warden et al, In Regulation of Body Weight: biological and behavioral mechanisms, C. Bouchard and G. A. Bray, eds. (West Sussex; John Wiley & Sons Ltd.), pp. 285-305). Combinatorial approaches can also be used to ensure that the UCP2 coding sequence is activated in the target tissue (Butt et al, Gene Exp. 4:319 (1995); Hart, Semin. Oncol. 23:1521 (1996)).

Antisense oligonucleotides complementary to UCP2 mRNA can be used to selectively diminish or ablate the expression of the protein, for example, at sites of inflammation. More specifically, antisense constructs or antisense oligonucleotides can be used to inhibit the production of UCP2 in high expressing cells (spleen, thymus, leucocytes, bone marrow and stomach). Antisense mRNA can be produced by transfecting into target cells an expression vector with the UCP2 gene sequence, or portion thereof, oriented in an antisense direction relative to the direction of transcription. Appropriate vectors include viral vectors, including retroviral, adenoviral, and adeno-associated viral vectors, as well as nonviral vectors. Tissue specific promoters can be used (e.g. leptin gene promoter or aP2 gene promoter specific for adipose cells, muscle creatine kinase promoter specific for skeletal muscle and lymphoid cell promoters). Alternatively, antisense oligonucleotides can be introduced directly into target cells to achieve the same goal. (See also other delivery methodologies described above in connection with gene therapy.) Oligonucleotides can be selected/designed to achieve a high level of specificity. (See also Matteucci et al, Nature 384:20 (1996)).

It has been recently demonstrated that increased O₂ consumption associated with cachexia of malignancy can be attenuated by indomethacin, a cyclooxygenase inhibitor. This is thought to be due to inhibition of prostaglandin production (Roe et al, Metabolism 46:359 (1997)). Thus, agents that block UCP2 expression and/or activity can be expected to be useful in the treatment of cachexia.

The therapeutic methodologies described herein are applicable to both human and non-human mammals (including cats and dogs).

Certain aspects of the present invention are described in greater detail in the non-limiting Examples that follow.

### EXAMPLES

The following experimental methodologies relate particularly to Examples I and III-VII that follow.

Yeast transfections and functional analysis using flow cytometry. The diploid yeast (Saccharomyces cerevisiae) strain W303 (a/α; ade2-10; his 3-11-15; leu2-2, 112; ura3-1; can1-100; try-Δ1) was used for expression of UCP2. The pYedP-UCP2 or UCPmut expression vectors were introduced into yeast by electroporation and transformants were selected for uracil auxotrophy as described by Bouillaud et al (EMBO J. 13:1990 (1994)). Expression of the UCP2, UCP1 or UCPmut under the control of the gal-cyc promoter was induced by galactose in the absence of glucose. Growth of yeast for flow cytometry analysis and measurement of mitochondrial membrane potential using the DiOC6(3) fluorescent probe (3,3′-dihexyloxacarbocyanine iodine, Molecular Probes, Eugene, Oreg.) are described by Bouillaud et al (EMBO J. 13:1990 (1994)).

Isolation of UCP2 CDNA clones. Human and mouse UCP2 IMAGE consortium clones (Washington University, St. Louis—Merck) identified in the EST database (http://www.ncbi.nlm.nih.gov) were purchased (Research Genetics, Huntsville, Ala.). Human UCP2 sequence was derived from IMAGE clones 129216, 158373, 163246 and 236034. GenBank accession for the human UCP2 coding sequence is U76367. Mouse UCP2 was also isolated independently (DR) by screening a mouse muscle cDNA library with rat UCP1 (GenBank accession U 69135). More than 20 mouse and human UCP2 clones are currently in the EST database, while only one UCP1 clone (IMAGE 529189) is represented in the EST database.

Mouse chromosomal mapping. The chromosomal position was determined by linkage mapping of restriction fragment length polymorphisms (RFLPs) in an interspecific cross as previously described (Watson et al, Mamm. Genome 2:158 (1992)). Hybridization of human UCP2 probe (892 bp insert of IMAGE clone 129216) defined EcoRI RFLPs that segregated in the strains used (C3H, 5.5 kb; M, spretus , 9.0 kb). Gene order was determined by analyzing all haplotypes and minimizing crossover frequencies. The reference loci in this interspecific cross that includes 2000 chromosomal markers are indicated with citations in an online database (http://www.informatics.jax.org/crossdata.html: select “seldin cross” and chromosome number 7).

### Example I

### Sequence and Uncoupling Activity of UCP2

The amino acid sequence of human UCP2 is 59% identical to human UCP1 (UCP2 sequence shown in FIG. 1A). The predicted coding sequence produces a protein of 309 amino acids with a molecular weight of 33,218 Daltons and an isoelectric point of 10.0. The amino acid sequence of mouse UCP2 (GenBank U69135) is 95% identical to human UCP2. Several protein motifs are conserved between UCP1 and UCP2. Both exhibit three mitochondrial carrier protein motifs, consistent with roles as ion transporters of the inner membrane, while the amino acids essential to ATP binding are also conserved.

To test the hypothesis that UCP2 is an uncoupling protein, yeast were transfected with UCP2 in an expression vector as previously reported for UCP1 (Bouillaud et al, EMBO J. 13:1990 (1994)). Rates of growth in liquid medium of transformed yeast were measured in the presence of galactose, which induces expression. Instantaneous generation times were compared after induction of vector, UCP2, control (empty) and UCP1 expression vectors. The values for UCP2 were 260±29 minutes (±SD, n=5), whereas the mean value for the strain transfected control vector (pYeDP vector) is 188±13 minutes (n=5, p<001 versus UCP2) and the mean value for UCP1 is 226±10 minutes (n=10, p<0.01 versus UCP2). These results indicate that UCP2 slows the growth of yeast more than does UCP1 (Bouillaud et al, EMBO J. 13:1990 (1994)).

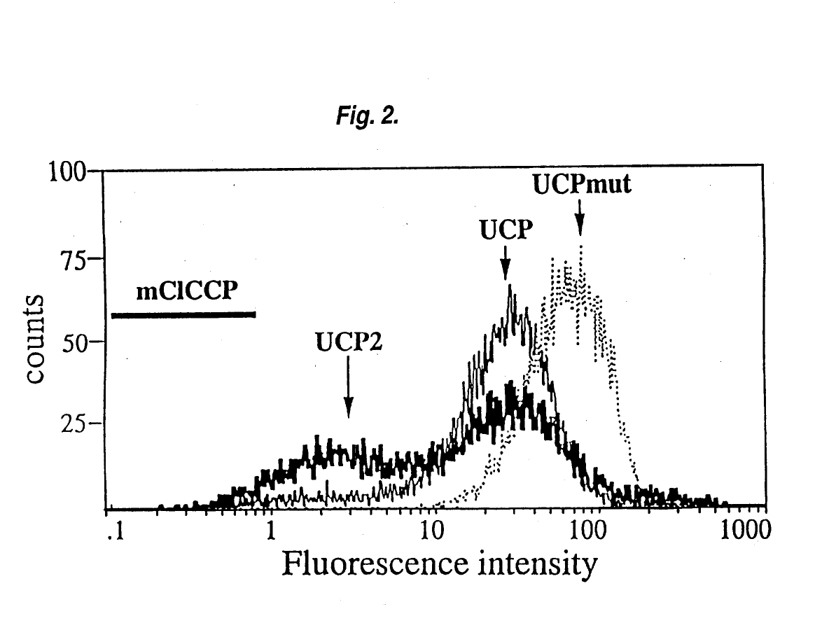

The in vivo decrease of membrane potential associated with uncoupling of respiration was analyzed by flow cytometry of yeast labeled with the potential sensitive probe DiOC6(3) . Using this procedure, it was previously demonstrated that UCP1 decreases the mitochondrial potential of transformed yeast (Bouillaud et al, EMBO J. 13:1990 (1994)). Mitochondrial potential is strongly decreased in strains containing either the UCP2 or UCP1 expression vectors, in contrast to strains containing empty or UCPmut (A36P/K150L mutations that inhibit UCP1 activity) expression vectors (FIG. 2). In the case of UCP2, 2 peaks were observed, one equivalent to cells expressing UCP1 and a second peak corresponding to a very low mitochondrial potential. Mean fluorescence intensities were: empty vector, 65.5; UCPmut, 43.0; UCP1, 22.5; UCP2, 15.5 and UCP2+mCICCP, 0.4 (FIG. 2). Very similar results were obtained in two additional experiments. Results of a representative experiment are shown in FIG. 2. These data show that UCP2 influences mitochondrial activity and can partially uncouple respiration.

### Example II

### Organization of Mouse and Human UCP2 Genes

A map showing the organization of the mouse UCP2 gene present in MMU2-L2. The mouse DNA inserted in lambda phage is 13.9 kb long and contains all the 8 exons and introns, and 5.3 kb of DNA upstream of the putative transcriptional start site (+1 site) (See FIG. 7) The DNA has been sequenced from −934 to +8600 bp (FIG. 8B). The region −246/+1 of the mouse UCP2 gene was placed in front of a CAT reporter gene. This CAT construct exhibited a promoter activity when transfected in cultured adipocytes (see Example X). A fragment of 1.8 kb located in the 5′ end of the gene (FIG. 8A) has also been sequenced.

A map showing the organization of the human UCP2 gene present in hUCP-g2. The human DNA inserted in lambda EMBL3 phage is 14 kb long (see FIG. 9). It contains all the 8 exons and introns, and a minimum of 3 kb of DNA upstream of the putative +1 site. Four regions, referred to as sequences 1, 2, 3 or 4 in FIGS. 1OA-D., have been sequenced. Sequence 1 corresponds to 640 bp of DNA forming the 5′ extremity of the human DNA. Sequence 2 correspcnds to a 1161 bp DNA from positions bp −511 to +650. This fragment contains the putative proximal human UCP2 promoter.

Human and mouse UCP2 genes have a very similar organization, exons 1 and 2 being noncoding exons, whereas exons 3-6 are coding exons.

### Example III

### Expression of UCP2 in Human and Mouse Tissues

To determine the possible sites of physiologic function of UCP2, Northern blot analyses were performed. UCP2 tissue distribution is markedly different from that of UCP1 (FIG. 3A). Probing of a multiple tissue northern blot from pooled adult tissues reveals UCP2 mRNA of 1.6 kb size present in skeletal muscle, lung, heart, placenta and kidney. In mice, UCP2 is expressed in brown adipose tissue (BAT) as well as white adipose tissue (WAT), and at high levels in heart and kidney. Probing of a Human mRNA Master Blot (Clontech, Palo Alto, Calif.) with UCP2 reveals that it is expressed at high levels in skeletal muscle, spleen, thymus, leukocytes, bone marrow and stomach. Low levels of UCP2 mRNA expression were observed in liver or brain.

### Example IV

### Mapping of UCP2 in Mice and Humans

Additional evidence for the role of UCP2 in obesity and diabetes was sought by determining the chromosomal position of UCP2. Whole genome mapping of mouse genetic crosses has identified at least 10 chromosomal loci influencing spontaneous and diet-induced obesity (Horvat et al, Genetics 139:1737 (1995); Collins et al, Mamm. Genome 4:454 (1993); Warden et al, J. Clin. Invest. 95:1545 (1995); West et al, J. Clin. Invest. 94:1410 (1994); Taylor et al Genomics 34:389 (1996); Seldon et al, J. Clin. Invest. 94:269 (1994)). Identification of the specific genes underlying these loci remains a challenge. A positional candidate approach, in which genes with suspected roles in obesity and insulin resistance are shown to have chromosomal loci co-incident with known obesity and diabetes loci, has been used to show that UCP2 is such a novel candidate gene. UCP2 (Ucp2) was mapped to mouse chromosome 7, tightly linked to the tubby mutation (FIG. 3B). Two independent sequence tagged sites derived from human UCP2 clones (Expressed sequence tags, EST 143230 and 226515) have been mapped to the homologous region of the long arm of human chromosome 11, between D11S916 and D11S911 (WI-1672 and WI-13873, Whitehead Institute Center for Genome Research radiation hybrid panel, http://www.ncbi.nlm.nih.gov/SCIENCE96/), consistent with mouse/human homology relationships suggesting an 11q13 chromosomal band location (DeBray et al, Genomics 33:337 (1996)).

### Example V

### UCP2 is a Positional Candidate Gene For Mouse and Human Obesity and Hyperinsulinemia Loci

The chromosomal mapping of UCP2 is co-incident with quantitative trait loci (QTLs) for obesity from at least three independent mouse models, one congenic strain, and human insulin dependent diabetes locus-4 (IDDM4) (Warden et al, J. Clin. Invest. 95:1545 (1995); Taylor et al, Genomics 34:389 (1996); Seldin et al, J. Clin. Invest. 94:269 (1994); Hashimoto et al, Nature 371:61 (1994)). Diet-induced obesity and diabetes have been demonstrated in the C57BL/6J (B6) mouse, while the A/J strain is resistant to the high-fat diet (Surwit et al, Diabetes 37:1163 (1988)). This syndrome is not due to inactivity (Brownlow et al, Physiology & Behavior 60:37 (1996)) or to hyperphagia (Surwit et al, Metabolism 44:545 (1995)) as B6 mice show higher feed efficiency than A/J mice (weight gained/calories consumed), when fed a high fat diet. Whole genome mapping has shown that a QTL for plasma leptin, insulin, and glucose is located on mouse chromosome 7, peaking near the tubby locus (Surwit et al, Diabetes 45:78A (1996)). Sequence analysis and mRNA expression levels suggest that tubby is not the underlying gene for this QTL. When Northern blots of white adipose tissue from A/J and B6 mice were hybridized with the UCP2 probe, mRNA levels were found to be higher in the obesity-resistant A/J than the obesity prone B6 (FIG. 4). In addition, UCP2 mRNA levels were significantly increased by the high-fat diet in A/J mice at early time points in the diet (days 7, 18 and 25), but only modestly in B6 animals (FIG. 3C). Core body temperature measured by a rectal temperature probe is also increased in A/J and B6 mice consuming the high fat diet, but to a greater extent (2×) in A/J than in B6 mice. These data are highly significant (n=10 for each group, p<0.01). However, after progression to severe obesity in the B6 mouse on a high fat diet for 18 weeks, levels of UCP2 were dramatically increased in both strains, but in contrast to early time points (7, 18, 25 days) levels of UCP2 were greater in the diet-induced obese B6 mice relative to the leaner A/J strain. Additionally, core body temperature increased more in A/J mice than in B/6 mice following the introduction of a high fat diet (p<0.05). Similar over expression of UCP2 is observed in adult, genetically obese ob/ob and db/db mice.

Regulation of UCP2 has been examined for factors that would regulate UCP1. In 11-week-old fed male Swiss mice, UCP2 mRNA was detected in BAT, WAT, thigh muscle and liver. Exposure at 4° C. or treatment with the β3 adrenergic agonist CL316,243 for 10 days did not influence UCP2 mRNA levels in these tissues. Finally, administration of leptin (2 μg, 2×/day for 4 days) failed to increased UCP2 expression. Leptin has been shown to increase sympathetic outflow to brown fat, thus potentially increasing UCP2 expression. Thus, UCP2 is regulated very differently than UCP1, which is induced by cold and β3 agonists. However, these data do not rule out the possibility that the sympathetic nervous system regulates UCP2.

### Example VI

### Increase in UCP2 Expression by Thiazolidinediones

Thiazolidinediones are known insulin-sensitizing agents that lower plasma glucose levels, and long chain fatty acids have been shown to be ligands for the PPAR (peroxisome proliferator activated receptor) family of receptors. Accordingly, a study was undertaken to determine whether agents that stimulate PPARgamma could increase expression of UCP2 in a model adipocyte cell line, HIB-1B.

HIB 1B cells were grown for 7 days in DMEM+10% charcoal-stripped serum (Media) or with the addition of the thiazolidine dione BRL 49653 (1 μM) and the RXRα ligand LGD-1069 (0.1 μM) (TZD/RXR). The results are the average of 2 independent experiments of 4 samples each. *, significantly different from Media samples, p<0.001. (See FIG. 6).

### Example VII

### Respiratory Rates

Respiratory rates have been measured in mitochondria isolated from yeast containing the empty pYeDP vector and the pUCP2 vector (Table 1). Upon addition of the FCCP uncoupler, the respiratory rate was increased by a factor of 7.1 in control mitochondria, while it was only increased by a factor of 4.3 in pUCP2 mitochondria (Table 1). This result clearly demonstrates that UCP2 has uncoupling activity. Addition of ADP stimulates respiration more in control mitochondria, again showing the UCP2 mitochondria are more uncoupled.

TABLE 1

| | Basal | Uncoupled | | ADP addition | |
|---|---|---|---|---|---|
| | rate | rate | fold | rate | fold |
| pYeDP | 14 | 100 | 7.1 | 33 | 2.3 |
| pUCP2 | 23 | 100 | 4.3 | 42 | 1.8 |

### Example VIII

### Role of UCP2 in a Mouse Model of Spontaneous Obesity

The Ucp2 gene is co-incident with a QTL for spontaneous multifactorial obesity in BSB mice. BSB mice are derived from a backcross of (C57BL/6J×Mus spretus) F1s×C57BL/6J. A locus with significant QTLs for body fat percent, hepatic lipase activity and plasma cholesterol is located on distal mouse chromosome 7 surrounding the tubby locus. mRNA extracted from the spleens of 16 BSB mice was probed on northern blots for UCP2 and actin. Levels of mRNA were quantitated by phosphorimager and UCP2 was normalized with actin. A negative correlation of UCP2/actin mRNA levels with body fat (p=0.016) and HDL cholesterol (p=0.023) was observed. The hypothesis that body temperature is correlated with body fat in BSB mice was also investigated. Core body temperatures were determined by rectal thermometers every week after weaning of 45 BSB mice. These BSB mice demonstrated a negative correlation of body temperature with percent body fat at sacrifice. P-values for the correlation of were p=0.018 at 6 weeks old (r²=0.12) and p=0.09 at week 8 (r²=0.10). These results indicate that UCP2 influences body fat percent by altering body temperature in a model of spontaneous obesity exhibited by mice maintained on a chow diet. (See Fisler et al, Obesity Res. 1:271 (1993); Warden et al, J. Clin. Invest. 92:773 (1993); Warden et al, J. Clin. Invest. 95:1545 (1995).)

### Example IX

### Common Sequence Polymorphisms of Human UCP2

Human mRNA was prepared from buffy coats (white blood cells) from 8 Hispanics who either have Type II diabetes or are the children of Type II diabetics. The complete human UCP2 coding sequence was amplified by RT-PCR of mRNA using primer pair 3 (hUCP2.cds3) as described in the Brief Description of FIG. 1E. The products were cloned into the Invitrogen pCR2.1 vector as described by the manufacturer. Sequencing was performed with 8 primers on each of the clones. Sequences were compared with the hUCP2 sequence submitted to Genbank (Accession No. U76367). 5 polymorphisms were observed in at least 2 of the 8 people sequenced. Each of these confirmed polymorphisms was also observed with all overlapping primers. 5 additional polymorphisms were observed with only one person or have conflicting data from different primer pairs. (See Table 2) Restriction enzymes have been identified that would differentially digest each of the alleles.

TABLE 2

| Polymorphism | 1 | 2 | 3 | 4 | 5 |
|---|---|---|---|---|---|
| base number in terms of | 445 | 502 | 508 | 869 | 913 |
| entire dna sequence | | | | | |
| amino acid number in terms | 34 | 53 | 55 | 104 | 190 |
| of open reading frame seq. | | | | | |
| hUCP2.genbank | L | A | A | K | N |
| Confirmed polymorphisms, | L | A | V | K | S |
| observed at least twice. | | | | | |
| Polymorphisms observed | P | V | V | K | S |
| only once. | | | | | |
| Short name | | | A55V | | N190S |
| Polymorphism | 6 | 7 | 8 | 9 | 10 |
| base number in terms of | 1052 | 1068 | 1142 | 1224 | 1262 |
| entire dna sequence | | | | | |
| amino acid number in terms | 236 | 242 | 266 | 294 | 306 |
| of open reading frame seq. | | | | | |
| hUCP2.genbank | D | Y | P | L | E |
| Confirmed polymorphisms, | D | Y | P | M | E |
| observed at least twice. | | | | | |
| Polymorphisms observed | E | N | P | M | E |
| only once. | | | | | |
| Short name | | | | L294M | |

Restriction enzymes of HUCP2.genbank for polymorphism analysis:

Polymorphism 1: Frequency of base change T to C at 445 of enire expressed is 77% and 33% C. Amino acid change occurs in polymorphisms observed only once from L to P.

site=t/ccgga, possible enzymes=AccIII, BseAI, BseAI, BsiMI, Bsp13I, BspEI, Kpn2I, MroI, There are no other areas where this enzyme would cut.

Polymorphism 2: Frequency of base change C to T at 502 of entire expressed sequence is 22% C, 88% T; base change T to A at 503 of entire expressed sequence is 64% T, 36% A. Amino acid change occurs in edited all sequence from A to V. Restriction enzyme cleavage site=/gtnac, possible enzyme is MaeIII. Other areas that this enzyme cuts are: 119, 583, 865.

Polymorphism 3: Frequency of base change C to T at 508 of entire expressed sequence is 57% C and 43% T as shown in 2 primers. Amino acid chance occurs in edited and edited all sequences from A to V. Restriction enzyme cleavage site=gcn/ngc, possible enzyme is Cac8I. Other areas that this enzyme cuts are: 58, 70, 153, 250, 254, 336, 340, 364, 670, 756, 901.

Polymorphism 4: Frequency of base change C to T at 869 of entire expressed sequence is 73% C and 27% T, shown in 2 primers. Seventy-five percent of examined individuals are T. No amino acid change occurs at this possible polymorphism site. Restriction enzyme cleavage site=cctc, possible enzyme is MnlI. Other areas that this enzyme cuts are: 97, 231, 278, 357, 546, 594, 609, 624, 639, 695, 796, 824, 891.

Polymorphism 5: Frequency of base change A to G at 913 of entire expressed sequence is 80% A and 20% G as shown in two primers. Twenty-five percent of examined individuals are G. Amino acid change occurs in edited and edited all sequences from N to S. Restriction enzyme cleavage site=cmg/ckg where m=a,c and k=g,t; possible enzymes=MspA1I, NspBII or site=cag/ctg with enzyme PvuII. Other areas that cmg/ckg cuts are:cat/ctg: 65, 877; ccg/cgg: 177.

Polymorphism 6: Base change C to A at 1052 of entire expressed sequence is included in edited all seauence because 4 out of 8 exhibit A in primer −21M13 and C in primer 241.1. Amino acid change occurs in edited all sequence from D to E. Restriction enzyme cleavage site: gacn/nngtc, possible enzymes are AspI, AtsI, Tth111I. There are no other areas where this site occurs.

Polymorphism 7: Base change T to A at 1068 of entire expressed sequence is included in edited all sequence because 5 out of 8 exhibit A in primer −21M13 and T in primer 241.1. Four out of five individuals are the same ones changing as in Polymorphism 6. Amino acid change occurs in edited all sequence from Y to N. There are no enzymes to cleave near this possible polymorphism.

Polymorphism 8: Frequency of base change C to T at 1142 of entire expressed sequence is 64% C and 36% T as shown in 4 primers. No amino acid change occurs at this possible polymorphism site. Restriction enzyme cleavage site=t/cga, possible enzymes are TagI and TthHB8I. There are no other areas where this site occurs.

Polymorphism 9: Frequency of base change C to A at 1224 of entire expressed sequence is 64% C and 36% A as shown in 4 primers. The same individuals have the change from the expressed sequence as in Polymorphism 8. An amino acid change occurs in edited and edited all sequences from L to M. Restriction enzyme cleavage site=gcagc, possible enzymes are BbI and Bst71I. Other areas that this enzyme cuts are 258 and 368.

Polymorphism 10: Frequency of base change G to A at 1262 of entire expressed sequence is 57% G and 43% A. Six out of seven exhibit A. No amino acid change occurs at this possible polymorphism site. Restriction enzyme cleavage site=cctc, possible enzyme is Mn1I. The enzyme cleaves the complementary DNA but not the hUCP2.GenBank sequence at this site. Other areas that this enzyme cuts are 97, 231, 278, 357, 522, 536, 594, 609, 624, 639, 695, 796, 824, 891.

Amplification of human UCP2 exons containing polymorphisms.

The following primers are designed to amplify hUCP2 exons 4, 6, 7 and 8 from genomic DNA. Common amino acid variants are present in exons 4, 6, and 8: A55V is in exon 4, N190S is in exon 6, and L294M is in exon 8. N190S is expected to alter a PvuII site. It is expected that Ser 190 will cut and Asn190 will not cut with PvuII. Primers for exon 7 have also been designed.

### Results

Exon 4: Both primer pairs 1 and 2 produce single bands of the expected size.

Exon 6: Primer pair 2 (3219 - 3399) produces a single band of the expected size. Primer pair 1 (3147- 3416) did not work.

Exon 7: Primer pair 1 (4316-4594) produces a single band of the expected size. Primer pair 2 (4284 - 4598) did not work.

Exon 8: Primer pairs 1 and 2 both produce multiple bands, including ones of the expected size.

PCR conditions: Clontech KlenTaq plus polymerase is used. PCR conditions were 94° C. 1 min, 94° C. 30 sec, 66° C. 30 sec, 68° C. 30 sec, repeat steps 2-4 29-times, 4° C. indefinate. Reaction volume was 25 μl in an MJ research PTC-200.

======================================================
amplifies bp 1794-2155 of hUCP2g (Meudon)
hUCP2g.e4f1

| | |
|---|---|
| Forward Oligo: | 5′> TGTCTACTCT GTTCCCTCCC C <3′ |
| >>Length: | 21 |
| >>Melting Temperature: | 60.5 |
| >>Max AT Run Length: | 2 |
| >>GC Clamp Strength: | 57 |
| >>% GC Content: | 57 |
| >>Hairpin Stem: | 1 |
| >>Primer Dimer: | 4 |
| >>No Palindromes. | |
| hUCP2g.e4r1 | |
| Reverse Oligo: | 5′> GGCCTACACC CTTGCTCC <3′ |
| >>Length: | 18 |
| >>Melting Temperature: | 60.6 |
| >>Max AT Run Length: | 2 |
| >>GC Clamp Strength: | 45 |
| >>% GC Content: | 67 |
| >>Hairpin Stem: | 2 |
| >>Primer Dimer: | 6 |
| >>Longest palindrome has 4 bases (bases 1 to 4) | |
| >>Product Size: | 362 |
| >>Annealing Temperature: | 60.7 (Try T = 55.5) |

======================================================
amplifies bp 1858-2281 of hUCP2g Meudon
hUCP2g.e4f2

| | |
|---|---|
| Forward Oligo: | 5′> TGACTGGAGG TGGGAAGG <3′ |
| >>Length: | 18 |
| >>Melting Temperature: | 59.6 |
| >>Max AT Run Length: | 2 |
| >>GC Clamp Strength: | 47 |
| >>% GC Content: | 61 |
| >>Hairpin Stem: | 1 |
| >>Primer Dimer: | 4 |
| >>No Palindromes. | |
| hUCP2g.e4r2 | |
| Reverse Oligo: | 5′> TATGTGGAGG ACCAGGGC <3′ |
| >>Length: | 18 |
| >>Melting Temperature: | 59.4 |
| >>Max AT Run Length: | 3 |
| >>GC Clamp Strength: | 60 |
| >>% GC Content: | 61 |
| >>Hairpin Stem: | 2 |
| >>Primer Dimer: | 6 |
| >>No Palindromes. | |
| >>Product Size: | 424 |
| >>Annealing Temperature: | 59.9 (Try T = 54.4) |

======================================================
amplifies bp 3147-3416 of hUCP2g, Meudon
hUCP2g.e6f1

| | |
|---|---|
| Forward Oligo: | 5′> CAAGACCATT GCCCGAAG <3′ |
| >>Length: | 18 |
| >>Melting Temperature: | 60.2 |
| >>Max AT Run Length: | 3 |
| >>GC Clamp Strength: | 52 |
| >>% GC Content: | 56 |
| >>Hairpin Stem: | 2 |
| >>Primer Dimer: | 6 |
| >>No palindromes. | |
| hUCP2g.e6r1 | |
| Reverse Oligo: | 5′> ATGGGGGAAG GGTGAGAC <3′ |
| >>Length: | 18 |
| >>Melting Temperature: | 59.7 |
| >>Max AT Run Length: | 2 |
| >>GC Clamp Strength: | 14 |
| >>% GC Content: | 61 |
| >>Hairpin Stem: | 2 |
| >>Primer Dimer: | 4 |
| >>No Palindromes. | |
| >>Product Size: | 269 |
| >>Annealing Temperature: | 59.2 (Try T = 54.7) |

======================================================
amplifies bp 3219-3399 of hUCP2g, Meudon
hUCP2g.e6f2

| | |
|---|---|
| Forward Oligo: | 5′> TTCCTCCTCC CCGATACTC <3′ |
| >>Length: | 19 |
| >>Melting Temperature: | 59.0 |
| >>Max AT Run Length: | 3 |
| >>GC Clamp Strength: | 8 |
| >>% GC Content: | 58 |
| >>Hairpin Stem: | 2 |
| >>Primer Dimer: | 4 |
| >>No Palindromes. | |
| hUCP2g.e6r2 | |
| Reverse Oligo: | 5′> CCCAGCACCG TCTACCTC <3′ |
| >>Length: | 18 |
| >>Melting Temperature: | 59.2 |
| >>Max AT Run Length: | 2 |
| >>GC Clamp Strength: | 21 |
| >>% GC Content: | 67 |
| >>Hairpin Stem: | 2 |
| >>Primer Dimer: | 4 |
| >>No Palindromes. | |
| >>Product Size: | 180 |
| >>Annealing Temperature: | 58.1 (Try T = 54.0) |

======================================================
amplifies bp 4840-5338 of hUCP2g, Meudon
hUCP2g.e8f1

| | |
|---|---|
| Forward Oligo: | 5′> AAGTGGGAGG TGGAGGTG <3′ |
| >>Length: | 18 |
| >>Melting Temperature: | 58.4 |
| >>Max AT Run Length: | 2 |
| >>GC Clamp Strength: | 29 |
| >>% GC Content: | 61 |
| >>Hairpin Stem: | 0 |
| >>Primer Dimer: | 4 |
| >>No Palindromes. | |
| hUCP2g.e8r1 | |
| Reverse Oligo: | 5′> AGCTACAAGA GAGGAGGAGA CG <3′ |
| >>Length: | 22 |
| >>Melting Temperature: | 59.3 |
| >>Max AT Run Length: | 2 |
| >>GC Clamp Strength: | 30 |
| >>% GC Content: | 55 |
| >>Hairpin Stem: | 1 |
| >>Primer Dimer: | 4 |
| >>Longest palindrome has 4 bases (bases 1 to 4). | |
| >>Product Size: | 520 |
| >>Annealing Temperature: | 59.5 (Try T = 53.4) |

| | |
|---|---|
| ====================================================== | |
| amplifies bp 4831-5270 of hUCP2g, Meudon | |
| hUCP2g.e8f2 | |
| Forward Oligo: | 5′> CAGTGAGGGA AGTGGGAGG <3′ |
| >>Length: | 19 |
| >>Melting Temperature: | 60.7 |
| >>Max AT Run Length: | 2 |
| >>GC Clamp Strength: | 57 |
| >>% GC Content: | 63 |
| >>Hairpin Stem: | 1 |
| >>Primer Dimer: | 2 |
| >>No Palindromes. | |
| hUCP2g.e8r2 | |
| Reverse Oligo: | 5′> GGGGCAGGAC GAAGATTC <3′ |
| >>Length: | 18 |
| >>Melting Temperature: | 60.6 |
| >>Max AT Run Length: | 3 |
| >>GC Clamp Strength: | 19 |
| >>% GC Content: | 61 |
| >>Hairpin Stem: | 2 |
| >>Primer Dimer: | 6 |
| >>No Palindromes. | |
| >>Product Size: | 457 |
| >>Annealing Temperature: | 60.1 (Try T = 55.6) |
| ====================================================== | |
| amplifies 4316-4594 of hUCP2g, Meudon | |
| hUCP2g.e7f1 | |

| | |
|---|---|
| Forward Oligo: | 5′> CTTGCCTGCT CCTCCTTG <3′ |
| >>Length: | 18 |
| >>Melting Temperature: | 59.7 |
| >>Max AT Run Length: | 2 |
| >>GC Clamp Strength: | 35 |
| >>% GC Content: | 61 |
| >>Hairpin Stem: | 1 |
| >>Primer Dimer: | 6 |
| >>No Palindromes. | |
| hUCP2g.e7r1 | |
| Reverse Oligo: | 5′> GGTGGTTCTC TCCCACCC <3′ |
| >>Length: | 18 |
| >>Melting Temperature: | 60.3 |
| >>Max AT Run Length: | 2 |
| >>GC Clamp Strength: | 57 |
| >>% GC Content: | 67 |
| >>Hairpin Stem: | 3 |
| >>Primer Dimer: | 6 |
| >>No Palindromes. | |
| >>Product Size: | 278 |
| >>Annealing Temperature: | 60.4 (Try T = 54.7) |

======================================================
amplifies 4284-4598 of hUCP2g, Meudon
hUCP2g.e7f2

| | |
|---|---|
| Forward Oligo: | 5′> CCTTCTGGAA TGATGGGTG <3′ |
| >>Length: | 19 |
| >>Melting Temperature: | 58.9 |
| >>Max AT Run Length: | 3 |
| >>GC Clamp Strength: | 45 |
| >>% GC Content: | 53 |
| >>Hairpin Stem: | 2 |
| >>Primer Dimer: | 6 |
| >>No Palindromes. | |
| hUCP2g.e7r2 | |
| Reverse Oligo: | 5′> GAACTGGGTG GGGAGGAC <3′ |
| >>Length: | 18 |
| >>Melting Temperature: | 60.3 |
| >>Max AT Run Length: | 2 |
| >>GC Clamp Strength: | 26 |
| >>% GC Content: | 67 |
| >>Hairpin Stem: | 2 |
| >>Primer Dimer: | 6 |
| >>No Palindromes. | |
| >>Product Size: | 265 |
| >>Annealing Temperature: | 60.3 (Try T = 53.9) |

======================================================

### Example X

### Immune Cell Regulation of UCP2

(A) A tumor mast cell line, the Rat Basophilic Leukemia cell (RBL), is a model system for studies of immune system signal transduction. In the RBL cell, binding of an antigen to the IgE receptor on the cell surface results in the activation of phospholipase C-gamma (PLCg). This event leads to the activation of protein kinase C (PKC) and an increase in intracellular calcium which are required for degranulation and gene expression in RBL cells. Several drugs can mimic the intracellular events and degranulation occuring in this cell type in response to antigen activation of the IgE receptor. The inophore Ionomycin causes the release of calcium from intracellular calcium stores and the phorbol ester PMA is able to directly activate PKC. Both of these drugs are required to achieve maximal activation of RBL cells. In order to determine the expression level of UCP2 in RBL cells, these cells were treated with Ionomycin (5 mM), PMA (5 mM) or both drugs. A Northern blot of the RNA from these cells revealed a ˜4 fold induction of UCP2 expression in treated cells compared to untreated control cells. Addition of Ionomycin, PMA or both drugs simultaneously resulted in the same ˜4 fold increase in UCP2 expression. Since PMA and Ionomycin treatment do not cause an additive or synergistic expression of UCP2, maximal activation of RBL cells may not be required for UCP2 gene expression.

(B) Bacterial lipopolysaccharide (LPS) regulates UCP2 mRNA in macrophages. Bacterial LPS is known to induce many of the features of infection, including fever. Thioglycolate elicited mouse macrophage (peritoneal exudate cells, PEC) have been studied for regulation of UCP2 mRNA by LPS. It has been shown that PEC express UCP2 and that LPS decreases expression by several fold over a 24 hour time course. A study has also be made of UCP2 expression in cultured mouse macrophage RAW cells. These cells express UCP2. Addition of 100 ng/ml LPS in 5% calf serum decreases UCP2 mRNA levels by several fold after 24 hours. Lower doses of LPS are less effective, while shorter times produce smaller decreases. LPS seems to be more effective at decreasing UCP2 in cells maintained in 0.5% serum than in 5% serum. The LPS mediated decrease of UCP2 is not affected by indomethacin or by PGE2. All fo the macrophage northerns are normalized for actin expression and have been repeated at least once. These data show that LPS, which is known to influence immune function and to promote fever, can also influence immune function and to promote fever, can also inc.luence UCP2 mRNA levels. The results are consistent with a role of UCP2 in immune function.

### Example XI

### UCP2 Promoter Activity

A 246 bp putative promoter region for mouse UCP2 was cloned upstream of the chloramphenicol acetyl transferase gene in either the “sense” (also called “+” or “forward” orientation) or the “antisense” (or “−” or “reverse” orientation). When transfected into differentiated mouse HIB-1B adipocytes CAT activity (conversion of substrate into product) is observed for the “sense” (“+”) orientation, but not for the “antisense” (“−”) orientation. (See FIG. 11).

### Example XII

### The Linkage Study

Experimental Details:

Sample. A total of 640 individuals (299 males and 341 females) from 155 pedigrees were available for the present study. These were randomly ascertained families of French descent livina in the Québec city area and recruited to participate in the Québec Family Study, a population-based study of the genetics of physiological fitness and body composition. The age of individuals in the sample ranges from 18 to 94 years.

Phenotypes. BMI was obtained from height and weight measurements (BMI=weight in kg/height in m). FM, fat-free mass and %FAT were determined from body density measurements obtained by underwater weighing using the conversion factor of Siri (Adv. Biol. Med. Phys. 4:239 (1976)). RMR was determined by indirect calorimetry measurements using an open-circuit indirect calorimeter with the ventilated hood technique (Derioz et al, J. Clin. Invest. 93:838 (1994)). Measurements were taken in the morning after an overnight fast, while subjects sat quietly in a semireclined position for the 30 minute measurement period. The last 10 minutes were kept for calculation of the RMR. The VO₂ and VCO₂ data were converted into energy as recommended by Weir (J. Physiol. (Lond) 109:1 (1949)). The phenotypes were adjusted by sex, for age and age by regression procedures and RMR was further adjusted for FM and FFM. The residuals from the regressions were used for linkage analysis.

DNA typing. Genomic DNA was prepared from permanent lymphoblastoid cells by the proteinase K and phenol/chloroform technique. DNA was dialysed four times against TE buffer (10 mM Tris, 1 mM EDTA pH 8.0) for 6 hours at 4° C. and ethanol precipitated. Amplificarion (Easycycler, Ericomp, San Diego, Calif.) was done in 96 wells microliter plaques using 250 ng of genomic DNA 0.1 pmoles (D11S1321, D11S916) or 0.25 pmoles (D11S911) of the forward primer coupled to the infrared tag IRD41 (Licor) and, respectively, 0.1 or 0.4 pmoles of the reverse primer, 125 μM dNTP's, and 0.3 U Taq polymerase (Perkin Elmer, Roche Molecular Systems, Branchburg, N.J.) in PCR buffer (100 mM Tris-HCl pH 8.3, 15 mM MgCl₂, 0.5 M KCl, 0.01% gelatin) for a final volume of 10 μl. PCR cycles consisted of 1 cycle at 93° C. for 5 min., 10 cycles at 94° C. for 20 sec, 57° C. for 60 sec, and 24 cycles at 94° C. for 20 sec., 52° C. for 60 sec., except for D11S911 for which the first annealing temperature was set at 55° C., PCR products were analyzed on automatic DNA sequencer (Li-Cor) using 18 cm glass plates. Typing was done assisted by computer (One Dscan, Scannalytics).

Linkage analysis. Relative-pair based methods were used to test for linkage between the phenotypes and the marker loci. In the presence of linkage between a marker locus and a cuantitive trait (Y), relative pairs sharing a greater proportion of alleles identical by descent (Π) as the marker locus tend to have more similar phenotypes than pairs who share fewer alleles. Thus, under the hvpothesis of linkage, a negative relationship is expected between Π and the within pair variance. The sib-pair linkage method described by Haseman and Elston is the most widely used method to investigate linkage between a quantitative phenotype and a marker locus. This method has been extended to other types of relative pairs. Tests of linkage which combine the information from different types of relative pairs were developed and these tests have been shown to be more powerful than the Haseman and Elston method based only on sib-pairs. This relative pair linkage analysis has been implemented in the program RELPAL which considers the following five types of relative pairs: sibling, half-sibling, grandparent-grandchild, avuncular and first degree cousins. For each relative pair type, the statistic for testing linkage is obtained by dividing the estimated regression coefficient (β) by its standard error. Because the number (n) of relative pairs could vary among the different types of relatives depending on the complexity of the pedigrees, the contribution of each type of relative pairs needs to be weighted in the overall linkage statistic which combine information from all relative pairs. The linkage test implemented in RELPAL is:

$$T = \frac{{c}^{T} ß}{\sqrt{{c}^{T} Var ( ß ) c}}$$

where β is a vector containing the β's for each of the 5 types of relatives and c^{T} is a weighing vector based on n and the variance of Π and equal to: (Var(Πₛ)nₛ, Var(Πₕ)nₕ, Var(Π_{g})n_{g}, Var(Πₐ)nₐ, Var(Π_{c})n_{c}), where subscripts s, h, g, a and c stand for siblings, half-siblings, grandparent-grandchild, avuncular and first degree cousins, respectively. In the Québec Family Study, since there are no half-sibs, only sibling, avuncular, grandparental and cousin pairs were used in the relative-pair linkage analysis.

### Results

A study was undertaken to investigate the potential linkage relationships between three microsatellite markers which encompass the UCP2 gene location on 11q13 with resting metabolic rate (RMR), body mass index (BMI), percentage body fat (%FAT) and fat mass (FM) in 640 individuals from 155 randomly ascertained pedigrees from the Québec Family Study. Using a linkage analysis strategy based on sibling, avuncular, grandparental and cousin pairs, strong evidence of linkage was found between the marker D11S911 (p=0.000002) and RMR with more moderate evidence for D11S916 (p=0.006) and D11S1321 (p=0.02). Suggestive evidence of linkage was also observed between D11S1321 and %FAT (p=0.04) and FM (p=0.02). It is concluded that the three markers encompassing the UCP2 locus and spanning a 5 cM region on 11q13 are linked to resting energy expenditure in adult humans.

The mouse ucp2 gene was recently mapped to chromosome 7, closely linked to the tubby mutation, a mutation known to be responsible for the adult-onset obesity in this mouse model. Furthermore, the UCP2 mRNA level was found to be higher in the A/J mouse strain, which is resistant to diet-induced obseity, than in the obesity prone C5/BL/6J mouse. The evidence accumulated thus far on animal models indicates that the UCP2 gene plays a role in the development of obesity because of its role in energy metabolism. The human UCP2 gene has been mapped to chromosome 11q13 at a location distinct from tubby (11p15.1) but in the same chromosomal location as the Bardet-Biedl Syndrom locus (Online Mendelian Inheritance in Man, OMTM, Center for Medical Genetics, Johns Hopkins Univ., (Baltimore, Md.) and National Center for Biotechnology Information, National Library oC Medicine (Bethesda, Md.) (1986), WWW URL: http://www3.ncbi.nlm.nih.gov/omim/), a Mendelian Syndrome exhibiting obesity as one of its clinical features. UCP2 is also in the proximity (about 15 cM) of a locus (11q21-q22) recently uncovered through a genome-wide search and found to be linked to percent body fat in Pima Indians. To test the hypothesis that markers around the UCP2 gene may exhibit a linkage relationship with metabolic rate and body fat phenotypes, three markers (D11S916, D11S1321 and D11S911) were typed on 640 individuals from 155 pedigrees from the Québec Family Study. Linkage studies were undertaken with RMR, BMI, %FAT and FM using four types of relatives. RMR was adjusted for the effects of age, sex, FM and fat-free mass, whereas BMI, %FAT and FM were adjusted only for age and sex effects.

Table 3 presents the linkage results with the number of relative pairs available in each case. Strong evidence of linkage was observed between the D11S911 and RMR while more moderate evidence for linkage was found for the other two markers. As there were few pairs of first degree cousins, the same analysis was repeated after removing these pairs. In the latter case, the p-value for linkage between D11S911 and RMR (n=212 pairs) was reduced to p=0.001, while the one between D11S916 and RMR (n=279 pairs) went from p=0.006 to p=0.02. Evidence of linkage between D11S1321 and %FAT (p=0.04) and FM (p=0.02) was also found. No linkage was observed between the three markers and fat-free mass in this population. Overall, these results indicate that the UCP2 gene which is encoded within the 5 cM span covered by these markers, plays a role in determining resting energy expenditure in humans.

TABLE 3
Relative-pair linkage analysis of resting metabolic rate and
body fat variables with markers encompassing the UCP2 gene
in the Québec Family Study

| MARKER | HZ | cM | RMR | BMI | % FAT | FM |
|---|---|---|---|---|---|---|
| D11S916 | 0.72 | 78 | 0.006 | 0.27 | 0.50 | 0.11 |
| | | | (301) | (415) | (304) | (304) |
| D11S1321 | 0.64 | 79 | 0.02 | 0.36 | 0.04 | 0.02 |
| | | | (360) | (537) | (383) | (383) |
| D11S911 | 0.85 | 83 | 0.000002 | 0.38 | 0.23 | 0.26 |
| | | | (240) | (324) | (243) | (243) |

Based on four different types of relative pairs: siblings (165-275 pairs); avuncular (47-134 pairs); grandparental (45-94 pairs); first degree cousins (22-34 pairs). The entries are the p-values with total number of relative pairs given in parentheses.
HZ: heterozygosity; cM; distance in centimorgan.

Based on four different types of relative pairs: siblings (165-275 pairs); avuncular (47-134 pairs); grandparental (45-94 pairs); first degree cousins (22-34 pairs). The entries are the p-values with total number of relative pairs given in parentheses. HZ: heterozygosity; cM; distance in centimorgan. * * *

All documents cited herein are incorporated in their entirety by references.

What is claimed is:

1. A method of treating a disease or disorder associated with diminished UCP2 expression or activity comprising administering to an individual in need of such treatment an amount of an agent that enhances UCP2 expression or activity sufficient to effect said treatment.

2. The method according to claim 1 wherein said disease or disorder is obesity, diabetes, syndrome X, or hypothermia.

3. The method according to claim 1 wherein said disease or disorder is hyperinsulinemia or glucose intolerance.

4. The method according to claim 1 wherein said agent is an expression construct comprising a UCP2 encoding sequence or portion thereof that encodes functional UCP2.

5. A method of treating a disease or disorder associated with elevated UCP2 expression or activity comprising administering to an individual in need of such treatment an amount of an agent that inhibits UCP2 expression or activity sufficient to effect said treatment.

6. The method according to claim 5 wherein said disease or disorder is wasting, anorexia, inflammation, cachexia, fever, or hyperthermia.

7. The method according to claim 5 wherein said agent is an oligonucleotide that specifically hybridizes to UCP2 mRNA or an antibody, or binding fragment thereof, that is specific for UCP2.

8. A method of screening a compound for its ability to modulate UCP2 expression or activity comprising contacting a cell that expresses UCP2 with said compound under conditions such that modulation of UCP2 expression or activity can occur and measuring UCP2 expression or activity in the presence and absence of said compound and determining whether modulation is effected.

9. Isolated mammalian UCP2, or fragment thereof of at least 5 amino acids.

10. The UCP2 according to claim 9 wherein said UCP2 is human UCP2.

11. The UCP2 according to claim 10 wherein said UCP2 has the sequence shown in FIG. 1A.

12. An isolated nucleic acid sequence encoding mammalian UCP2, or portion thereof at least 15 nucleotides in length.

13. The nucleic acid according to claim 12 wherein said nucleic acid encodes human UCP2.

14. The nucleic acid according to claim 13 wherein said human UCP2 has the sequence shown in FIG. 1A.

15. A recombinant molecule comprising a vector and the nucleic acid according to claim 12.

16. A host cell comprising the recombinant molecule according to claim 15.

17. A method of making UCP2 comprising incubating the host cell according to claim 16 under conditions such that said nucleic acid is expressed and said UCP2 is thereby produced.

18. An antibody specific for UCP2 or antigen binding fragment thereof.

19. An antisense construct comprising a vector and a nucleic acid operably linked to a promoter, wherein said nucleic acid is oriented with respect to the promoter such that the transcript of the nucleic acid selectively hybridizes to UCP2 mRNA.

20. A host cell comprising the construct according to claim 19.

21. The cell according to claim 20 wherein said cell is a mammalian cell.
